(19) United States
(12) Patent Application Publication
(10) Pub. No.: US 2019/0134357 A1
(43) Pub. Date: May 9, 2019

(54) Apparatus For A Side-Loading Guidewire Torque Device

(71) Applicant: Philip Benz, Milwaukie, OR (US)

(72) Inventors: Anselmo Arratia, Spring, TX (US); Robert H. Niemeyer, III, Tigard, OR (US); Philip Benz, Portland, OR (US)

(73) Assignee: Semler Technologies, Inc., Milwaukie, OR (US)

(21) Appl. No.: 16/171,816

(22) Filed: Oct. 26, 2018

Related U.S. Application Data
(60) Provisional application No. 62/582,509, filed on Nov. 7, 2017.
(60) Provisional application No. 62/620,064, filed on Jan. 22, 2018.

(51) Int. Cl. A61M 25/09

(52) U.S. Cl. CPC A61M 25/09041; A61B 2017/00469

(57) ABSTRACT

The apparatus for a side-loading guidewire torque device is used by a clinician to manipulate a guidewire during medical procedures. The device is removably installed laterally onto the guidewire at any desired position along an exposed length of the guidewire. A convenient gripping surface of the device enables the clinician to easily grasp, advance, rotate and withdraw the guidewire during its deployment in a patient and easily remove, re-attach and reposition the device along the length of the wire after initial attachment.

THIS NON-PROVISIONAL APPLICATION CLAIMS BENEFIT OF PROVISIONAL PATENT APPLICATION No. 62/582,509, WHICH HAS A PRIORITY DATE OF Nov. 7, 2017, AND PROVISIONAL PATENT APPLICATION No. 62/620,064, WHICH HAS A PRIORITY DATE OF Jan. 22, 2018.

### FIELD OF THE INVENTION

The invention relates generally to the field of interventional medicine. More particularly, the invention relates to a device that a clinician removably attaches to a guidewire for the purpose of more easily grasping, torqueing, advancing and withdrawing it during deployment as part of a medical procedure.

### BACKGROUND OF THE INVENTION

This invention relates to guidewires used during medical procedures that are generally employed for navigating through internal passageways of a body, which can include the gastro-intestinal tract, the genito-urinary tract, and the vasculature, i.e. more particularly including blood vessels. The use of medical guidewires is well-known and examples of the fields of medicine commonly using such wires include cardiac catheterizations, interventional radiology and endoscopy.

During its deployment into a patient, the distal end of a guidewire is introduced into a body by a physician through an opening in the body, e.g. a puncture site into a blood vessel or a body orifice. Examples include a cardiac catheterization where the puncture site is located in either the radial, brachial or femoral artery or vein, or during an endoscopy procedure in a gastro-intestinal lab. For vascular punctures, often a cannula or sheath is first inserted into the puncture site and is used during the procedure for introducing guidewires, catheters and other equipment into the body. The clinician, usually a physician, grasps the guidewire and manipulates its tip through the sheath and the body to a target site by advancing it, often while twisting, or torqueing, it. Guidewires used in these procedures generally vary in length from 160 centimeters to over 250 centimeters. Generally varying in diameter from 0.014 inches to over 0.035 inches, the proximal end (i.e. that is grasped by the clinician) remains outside of the body. The guidewire tip is sometimes bendable and can be angled by the clinician to steer through curves in the body's passageways, often by rotating, or torquing, the wire while advancing or withdrawing it.

A catheter or other generally tube-shaped equipment having a lumen is then typically advanced over the deployed guidewire to the target site: the clinician inserts the proximal tip of the guidewire into the exposed distal end of the catheter, which has a hole that connects to the lumen that extends down the length of the catheter. The catheter is then advanced “over the wire” to the target site. In many procedures, more than one catheter is used, one after the other, using the same or another guidewire. When exchanging catheters, the existing catheter in the body is pulled out over the entire length of the deployed guidewire, and then replaced by a new catheter by advancing the new catheter over the wire.

Guidewires' external surfaces are generally smooth and sometimes lubricious, due to hydrophilic coatings designed to reduce friction during deployment. Thus, clinicians find that an auxiliary, optional device having a larger gripping area enables the wire to be more easily advanced or withdrawn, and to enable a twisting or torqueing rotation of the wire as needed to orient the distal tip inside the patient. Because catheter exchanges and guidewire exchanges are common in a single procedure, any such device must be sufficiently durable to withstand repeated attachment and detachment cycles while continuing to securely grasp the wire.

Existing partial solutions to this need include pin-vise grips, which are well known and continue in common use with medical guidewires as of the date of this present application. These generally comprise a cylindrical handle, a cap, and a soft metal collet embedded inside the handle, configured similarly to a small drill chuck with a cylindrical handle. This chuck is threaded over the proximal end of the guidewire and moved to a desired position. The cap, threadably attached to the handle, can then be tightened onto the wire by screwing it tight causing the chuck to compress the collet onto the wire so that the pin-vise device is firmly attached to the guidewire. Thereafter the pin-vise serves as a grip for grasping with fingers, and manually rotating (i.e. “torqueing”) and/or longitudinally moving the position of the guidewire within the patient's body.

One of the problems experienced with using pin-vise type grips is that they must be threaded onto the guidewire at its exposed proximal end. This is time-consuming and inconvenient because of the length of the guidewire and the small diameters of the wire and the lumens of the catheter and pin-vise grip.

Thus, the prior art includes other types of gripping or torqueing devices that attempt to overcome this problem. These involve various approaches to enabling side-loading of the wire into the device, instead of having to thread the wire into the torquing device.

Auth, in U.S. Pat. No. 4,829,999, describes a side-loading device that includes a cylindrical, flexible unitary body having a longitudinal slit and which is formed of an “elastic material” having a “large yield strain capacity” so that by pressing together two handles on the body, the longitudinal slit can be opened and placed onto the guidewire. When the handles are released, the resilient body, that does not include a separate spring, clamps the guidewire within the slit similarly to clamping a clothespin into a clothesline, i.e. with force applied by a living hinge, generally known and defined herein as a thin flexible or flexure bearing hinge made from the same material as the pieces it connects. This device overcomes the problem of having to thread the device onto the proximal end of the guide-wire, but the handles are located on the side of the body and may interfere with secure grasping and manipulation of the device, particularly when attempting to torque the wire.

Intlekofer, in U.S. Pat. No. 4,858,810 describes another side-loading device constructed from two parts assembled together to provide a pin-vise type of gripping handle for a guidewire that has an elongate cylindrical body having a slot along the length to receive the guidewire, with a “slider” mechanism or “thumbpiece” that moves within the slot and which, when pushed forward, tightens down upon the guidewire in order to secure it in the device. To sideload the wire, the device must be partially disassembled, then re-assembled following wire insertion.

Nelson, in U.S. Pat. No. 5,219,332 discloses a side-loading, generally tubular device that does not include a separate spring. It is composed of cylindrical socket and plug members that are adapted to fit together in a telescoping arrangement, and an elastomeric member that fits inside of the cylindrical members. Each cylindrical socket member includes a passageway that runs through its length, and a slot that also runs the length and extends from an outer surface of each cylindrical member to the passageways running therethrough. The elastomeric body member has one end anchored in one of the cylindrical members and the other end anchored in the other at the ends of the passageways. An elongated body of the elastomeric member is situated in the passageway running between the two ends. The two cylindrical members can be rotated following placement of the guidewire inside the slot to compress it against the elastomeric member to secure the wire within the device. Thus, an additional tightening step is required following the step of inserting the wire.

Boutillette, in U.S. Pat. No. 7,717,865 discloses several versions of a side-loading device, none of which include a separate spring. An area is provided in each embodiment in which to emplace a guidewire and several features are shown to secure it in place. One of these is a slider, which by moving against the wire, compresses it against the interior wall of the device, thereby holding it in place. In another embodiment, the device folds around the wire in the manner of a clamshell, presumably having a living hinge to enable the folding. In yet another embodiment, the device includes an interior channel, a tapered, flexible generally conical outer surface, a slit that opens into the channel, and a ring that moves along the tapered outer surface to provide graduated compression of the wire following its placement in the channel when the ring is moved from the narrower to the wider portion of the outer surface.

Nichols, in U.S. patent application Ser. No. 15/234,883 discloses a spring-driven, side-loading clamping device that requires a separate spring and a separate axle or pin to join the components together. The device is intended to secure guidewires for the purpose of easily enabling a clinician to manipulate them, and to enable one-handed attachment and detachment. From the application's abstract, Nichols describes:

“A device for manipulating a wire includes a first structure and a second structure pivotably mounted with respect to each other so as to define respective facing surfaces configured for alternating between a closed position and at least one open position, where each of the respective facing surfaces includes one or more detent elements and one or more tooth elements extending therefrom. In the device, the tooth elements of the first structure and the second structure are in an interlocking and overlapping arrangement in at least the closed position. Further, each of the tooth elements has a concave surface for defining a passage between the facing surface in the at least one open position. Additionally, each of the detent elements is arranged to face the concave surface of one of the tooth elements of an opposite one of the respective facing surfaces in the closed position.”

The two structures, termed a first comb structure and second comb structure, are joined together using a pin that passes through pin holes or grooves in the comb structures and use a spring element to apply closing force to push these structures together. Tooth elements and detents are present on each comb structure and form the means for securing a guidewire. As Nichols describes in paragraph 0037, “the plurality of detents and the plurality of tooth elements are arranged so that, in the closed configuration, the concave surfaces for the plurality of tooth elements of each one of the first and the second comb structures rests on or contacts an innermost edge on one of the plurality of detents for the other one of the first and the second comb structures.” Further, as Nichols describes in paragraph 0039, “the shape and locations of the detents and the facing surfaces of the tooth elements are selected so that in the closed position, the innermost edge of each of the detents in one comb is in contact with a portion of the concave surfaces of the tooth elements of another comb while reducing the size of passage as much as possible. As a result, a wire in passage receives even pressure from both sides along its entire length.”

Boston Scientific marketed a device in conjunction with its Rotablator™ rotational atherectomy system called wireClip™ Torquer, that permits side-loading a stainless steel guidewire into the device, where it is held in place by facing surfaces that include an abrasive material. Because of likelihood of damaging coatings, this would not be used with coated guidewires. Its name notwithstanding, the common use of the wireClip was to serve as a brake on the Rotablator atherectomy wire, to prevent its rotation during deployment and use of the atherectomy system, thus its purpose and use differs from that of the present invention. It featured two c-shaped springs and an abrasive surface along its entire interior length, all of which length maintains contact with the wire, to enable better wire retention. Applicant has not found a patent corresponding to the wireClip Torquer device.

### SUMMARY AND OBJECTS OF THE INVENTION

The apparatus for a side-loading torque device disclosed herein includes at least two frames and a spring, assembled so that a clinician can grasp a guidewire, move it along its longitudinal axis, and rotate, or torque, it during deployment in a patient. Additional features of the assembled device efficiently facilitate the benefits described above.

Each rigid, non-elastic and non-flexible frame includes at least one selected from the following group of elements: a clamping body member, release handle, spinner handle, spring slot, clamping pad, backstop, wire entry guide, wire captivating notch, ribs, pivot tab and mating pivot cavity, and elongate pivot bar and mating elongate pivot socket. Pivot tabs and pivot cavities having different forms may be present on a frame; further a first frame may include only a pivot tab and a second frame may include only a pivot cavity, or a single frame may include both, or in a combination of elements to form an interlocking key pivot. Similarly, elongate pivot bars and elongate pivot sockets having different forms may be present on a frame; further a first frame may include only a pivot bar and a second frame may include only a pivot socket, or a single frame may include both, or in a combination of elements to form an interlocking key pivot. When the frames are assembled into a device, the following assembly elements may be formed: i) the pivot bar and pivot socket mate to form an interlocking pivot mechanism to enable pivoting, i.e. pivotable movement; ii) the pivot tab and pivot cavity mate to form a centric locking mechanism that prevents lateral and longitudinal movement and movement orthogonal to the longitudinal axis of the frames relative to each other while enabling pivoting, i.e. pivotable movement of the frames; iii) a pivot tab, pivot cavity, pivot bar and pivot socket are combined to form an interlocking key pivot to secure the frames, enable pivoting, i.e. pivotable movement of the frames, and prevent lateral and longitudinal movement and movement orthogonal to the longitudinal axis of the frames relative to each other; iv) a clamping body is composed of the clamping body members; v) a spinner element is composed of the spinner handles; vi) a wire clamp or a wire captivating clamp is composed of the clamping pads. Some or all of these assembly elements, in varied combinations, may be included in an assembled torque device of the present invention. The wire captivating clamp, which includes a wire captivating notch, may be included in the side-loading torque device instead of or in addition to a wire clamp. The wire captivating clamp provides mechanical obstacles to enhance securement of a retained guidewire by helping prevent unintentional movement or dislodgement during its deployment.

At least one resilient member, otherwise and more commonly known as a spring, which may be in a “C” shape and known as a C spring, provides i) a joining of the apparatus's two frames together such that the assembled torque device opens and closes in a pivotable movement, and ii) the clamping force that holds the guidewire in place.

Because of the interlocking pivot mechanism, centric locking mechanism, and interlocking key pivot that, individually or in combination, and together with the spring, join the two frames and permit pivotable movement, the torque device has a hinge but has no requirement for a pin, axle, or pinhole or living hinge for securing the frames together, thus these are not included in the torque device of the present invention. A “hinge” is generally known and herein defined as “a jointed device or flexible piece on which a door, gate, shutter, lid, or other attached part turns, swings, or moves.”

A clamping mechanism of a torque device that has a relatively large wire-contacting area, at a given force, distributes applied force, thus resulting in a decrease in local pressure (which varies inversely with wire-contacting area). A relatively larger wire-contacting area, with a given spring, reduces the ability of the clamping pad to retain the wire in the device and reduces the ability to apply torque, which increases the opportunity for dislodgement, disengagement or rotational slippage during use. A smaller, or limited-width, wire-contacting area enables use of a smaller spring than would otherwise be required while decreasing opportunity for disengagement or rotational slippage. This enables lower cost of material, forming, and assembly, and easier user operation.

The torque device of the present invention minimizes the guidewire-contacting area of the torque device so as to achieve increased normal force and local pressure with a given spring, therefore: i) tooth and detent elements in a comb element for capturing the guidewire are also not included since, in the prior art, these provide relatively large wire-contacting area, and ii) abrasive surfaces on the interior wire-contacting surfaces are not included since these damage coatings on guidewires and are not necessary in the present invention for retaining the guidewire during deployment. Because limited-width clamping pads securely capture the guidewire with the force applied by the spring, there are also no channel, passageway, collet, ferrule, slider, body or plug member, or pin-vise elements included in the present invention. Further, because a spring is included in the present invention that enables durability sufficient to withstand the repeated deployment cycles normal for interventional procedures, there is no need for a living hinge, the durability of which degrades with repeated use, and such is not included. Notably, a torque device clamping mechanism featuring a living hinge formed of a non-rigid thermoplastic risks undesirable changes in applied force and ease of deployment with repeated use. Further, such thermoplastics having the ability to form a living hinge are likely to deflect by conforming to the wire's circumference under compression (i.e. with application of clamping force), resulting in greater wire-contacting area and reduced normal force and reduced local pressure.

The torque device of the present invention thus distinguishes itself from the prior art at least and in particular by virtue of: i) the limited size, or limited-width, of the wire-contacting area; ii) a clamping means that includes a wire clamp or a wire captivating means that includes a wire captivating clamp that includes a wire captivating notch; iii) hinging securement and pivoting means that includes at least the interlocking pivot mechanism, interlocking key pivot, or the centric locking mechanism, or a combination of these, that obviates the need for a pin or axle, a hole for these to pass through, and a living hinge, thus more simply enabling assembly and hinging and pivoting, as shall be further described herein.

Objects of the present invention include enabling: i) a clinician to repeatably and quickly attach and detach a torque device from a guidewire without damaging the guidewire or coatings on the guidewire, for example, by abrasion from an interior wire contacting surface of the device; ii) easy, quick positioning of the device at any exposed location on the wire without having to thread it over the exposed proximal end of the wire; iii) secure retention of the guidewire to avoid unintentional dislodgement; iv) a clinician to securely grasp and manipulate a guidewire during its deployment in a patient, more particularly by advancing and withdrawing it while rotating, or torquing, it; v) rapid, one-handed removal from a guidewire; vi) efficient manufacture by minimizing materials, component parts, and assembly steps and time. As is demonstrated below, the hinging, spring-driven device of the present invention achieves all of these objects and demonstrates advantages over the prior art.

### BRIEF DESCRIPTION OF THE DRAWINGS

FIG. 1a is a perspective view of an embodiment of an assembled torque device in use, holding a guidewire.

FIG. 1b is an end view, with parts removed for clarity, taken along lines 1b-1b of FIG. 1a of an assembled torque device in use, holding a guidewire.

FIG. 1c is a perspective view of the interior of a first frame component of the torque device.

FIG. 1d is a perspective view of the interior of a second frame component of the torque device.

FIG. 2a is a perspective view of an assembled torque device with a guidewire, held open.

FIG. 2b is an end view taken along lines 2b-2b of FIG. 2a of the assembled torque device with a guidewire, held open.

FIG. 3 is a side view taken along lines 3-3 of FIG. 2a showing an assembled torque device with a guidewire, held open.

FIG. 4 is a perspective view of an alternative embodiment of an assembled torque device, held open.

FIG. 5 is a perspective exploded view of an alternative embodiment torque device.

FIG. 6a is an end partly sectioned view of a centric locking mechanism of the assembled torque device.

FIG. 6b is a side partly sectioned view of a centric locking mechanism of the assembled torque device.

FIG. 6c is a perspective view of an interior view of a part of a spinner of a torque device.

FIG. 6d is a perspective view of an interior view of a part of a spinner of a torque device that faces the spinner of FIG. 6c.

FIG. 7a is a perspective view an interlocking pivot of an assembled torque device.

FIG. 7b is an end view taken along lines 7b-7b of FIG. 7a of an interlocking pivot of the assembled torque device.

FIG. 7c is a perspective view, with parts removed for clarity, of an interlocking key pivot of an assembled torque device that includes the spinners of FIGS. 6c and 6d.

FIG. 8a is a top view showing a frame of a torque device with a guidewire.

FIG. 8b shows an end partly sectioned view with parts removed for clarity, taken along lines 8b-8b of FIG. 8a, of the assembled torque device holding a guidewire.

FIG. 9a is a side perspective view of an assembled torque device.

FIG. 9b is a bottom perspective view of a second frame of a torque device.

FIG. 9c is a partly sectioned end view, with parts removed for clarity, of an assembled torque device taken along lines 9c-9c of FIG. 9a.

FIG. 9d is an end view, with parts removed for clarity, of a wire captivating clamp of an assembled torque device, taken along lines 9d-9d of FIG. 9a.

FIG. 9e is an end view, with parts removed for clarity, of a wire captivating clamp of an assembled torque device, which holds a guidewire, taken along lines 9d-9d of FIG. 9a.

FIG. 9f is an end view, with parts removed for clarity, of another embodiment of a wire captivating clamp of an assembled torque device, which holds a guidewire, taken along lines 9d-9d of FIG. 9a.

FIG. 10a is a perspective side view of an assembled torque device held open.

FIG. 10b is an end view of the assembled torque device taken along lines 10b-10b of FIG. 10a, held open.

FIG. 10c is a perspective view of an assembled torque device.

### DETAILED DESCRIPTION OF THE PREFERRED EMBODIMENTS

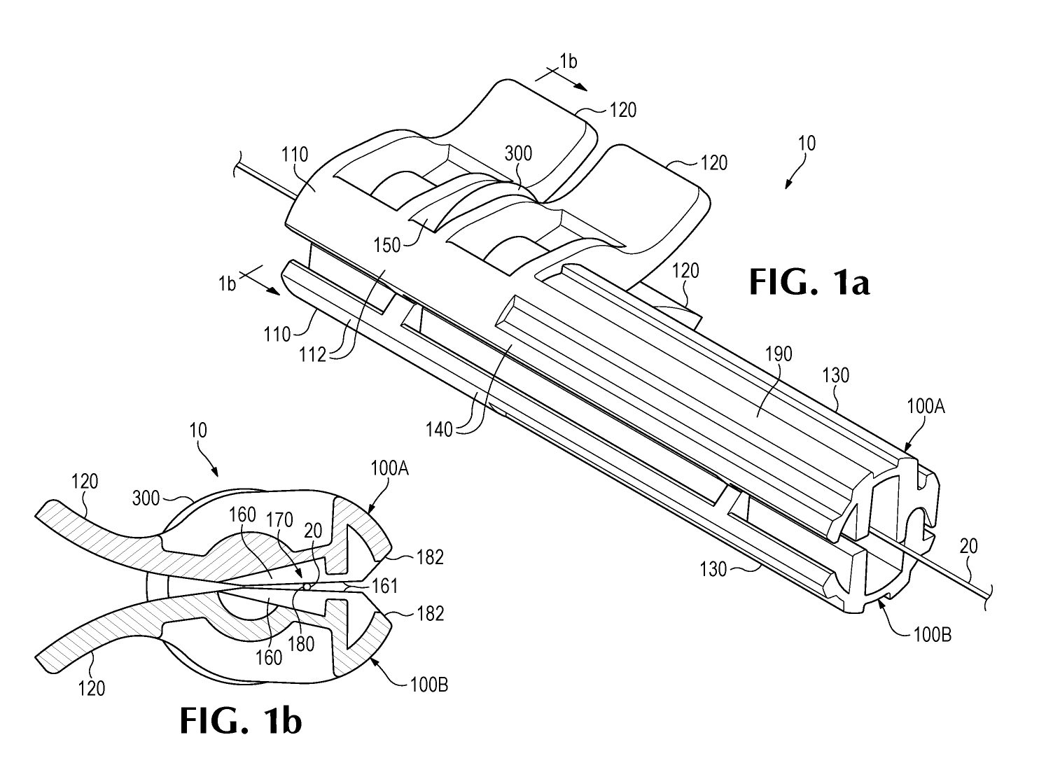

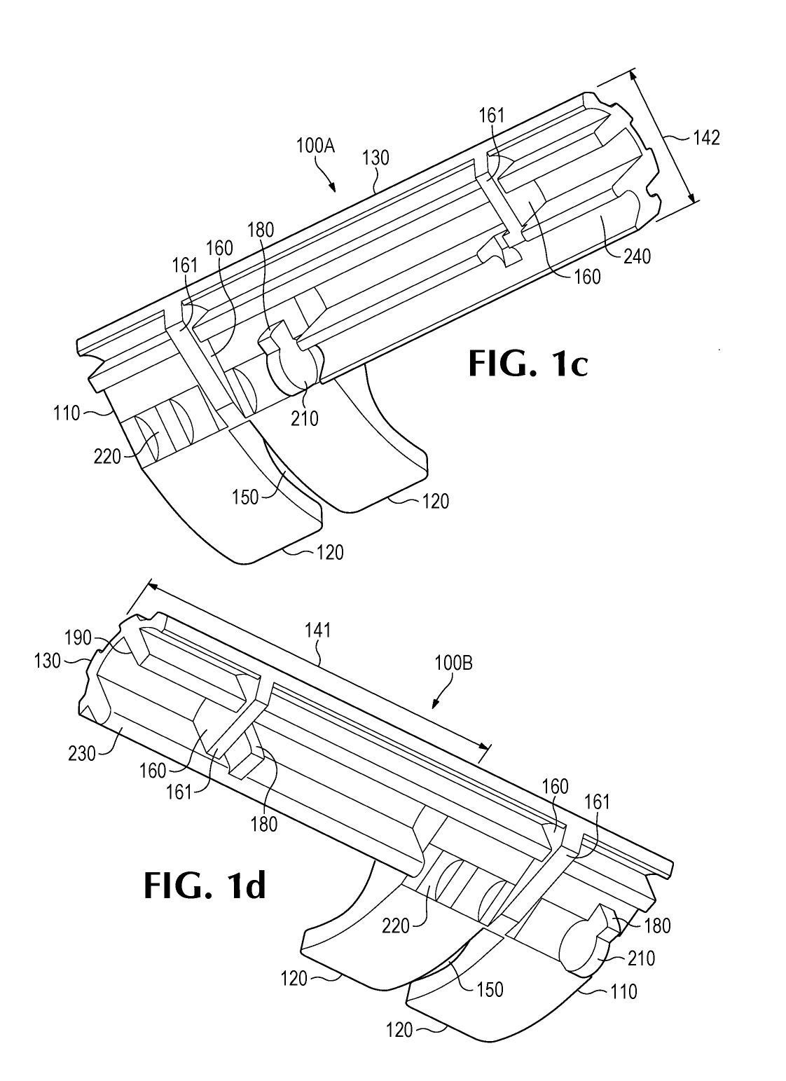

The apparatus for a side-loading torque device 10 is shown in use, clamping a guidewire 20 in the perspective view of FIG. 1a. The torque device 10, which has two ends, includes a first frame 100A and second frame 100B, and a spring 300. Each of the frames 100A and 100B includes a clamping body member 110, to which are attached flat release handles 120 and a single spinner handle 130, and longitudinal ribs 190 on the exterior surfaces of the spinner handle 130. The release handle 120 includes a spring slot 150, in which a C spring 300 is placed. The assembled torque device 10 of this embodiment includes a single spinner 140, or a “first spinner,” that is composed of the spinner handles 130, and a clamping body 112 that is composed of the clamping body members 110. The spinner 140 and clamping body 112 of the device 10 are formed when the frames 100A and 100B are assembled together and secured with a spring 300. Alternative embodiments of the torque device 10 may include more than one spring slot 150 or release handle 120 or spring 300 or spinner handle 130. Only a section of guidewire 20 is shown in the Figures for ease of viewing.

The apparatus for a side-loading torque device 10 is shown in use, clamping a guidewire 20 in the end view of FIG. 1b. The torque device 10 includes a first frame 100A and second frame 100B, and a spring 300. Each of the frames 100A and 100B has an interior and an exterior surface and includes a wire entry guide 182, which more easily permits a guidewire 20 to be inserted into the clamping surfaces 161 of the wire clamping pads 160, by providing an opening that funnels the wire 20 into the proper position. A wire clamp 170 is comprised of the facing wire clamping pads 160 and included clamping surfaces 161. More particularly, the guidewire 20 is held in place between the clamping surfaces 161 and against backstop 180, by the clamping force exerted by the spring 300. The torque device 10 has at least two sides, in particular a closed side that does not open, i.e. the side on which the spring 300 is inserted into the spring slot 150, and an opening side that opens to accept a guidewire 20 and which includes the wire entry guide 182. Also shown in FIG. 1b are the handles 120 and wire clamp 170 composed of the clamping pads 160 and included clamping surfaces 161.

Features of a first frame 100A are shown in the perspective view of FIG. 1c and include: clamping body member 110, release handles 120, spinner handle 130, spring slot 150, wire clamping pads 160 that include clamping surfaces 161, a pivot cavity 220 and elongate pivot socket 240. In addition, a backstop 180 is present on the pivot tab 210. The spinner handle 130 has a width 142.

Features of a second frame 100B are shown in the perspective view of FIG. 1d and include: clamping body member 110, release handles 120, spinner handle 130, spring slot 150, wire clamping pads 160 that include clamping surfaces 161, a pivot cavity 220 and elongate pivot bar 230. In addition, a backstop 180 is present on the pivot tab 210 and another is present on the spinner handle 130. The spinner handle 130 also includes a longitudinal rib 190 on its interior, and has a length 141.

Frames 100A and 100B matably fit together, with a spring 300, to form the assembled device 10. Alternative embodiments of the frames 100A and 100B may each include different numbers or locations of the clamping pads 160, backstops 180 on the clamping body members 110 and spinner handles 130, spring slot 150, or spring 300. Alternative embodiments may include more than one of the pivot tab 210, pivot cavity 220, pivot bar 230, or pivot socket 240 and these may be located on the opposite frames or take shapes or relative dimensions or be configured differently from those shown in the drawings. Also, alternative embodiments may exclude i) the pivot tab 210 and pivot cavity 220, or ii) the pivot bar 230 and pivot socket 240.

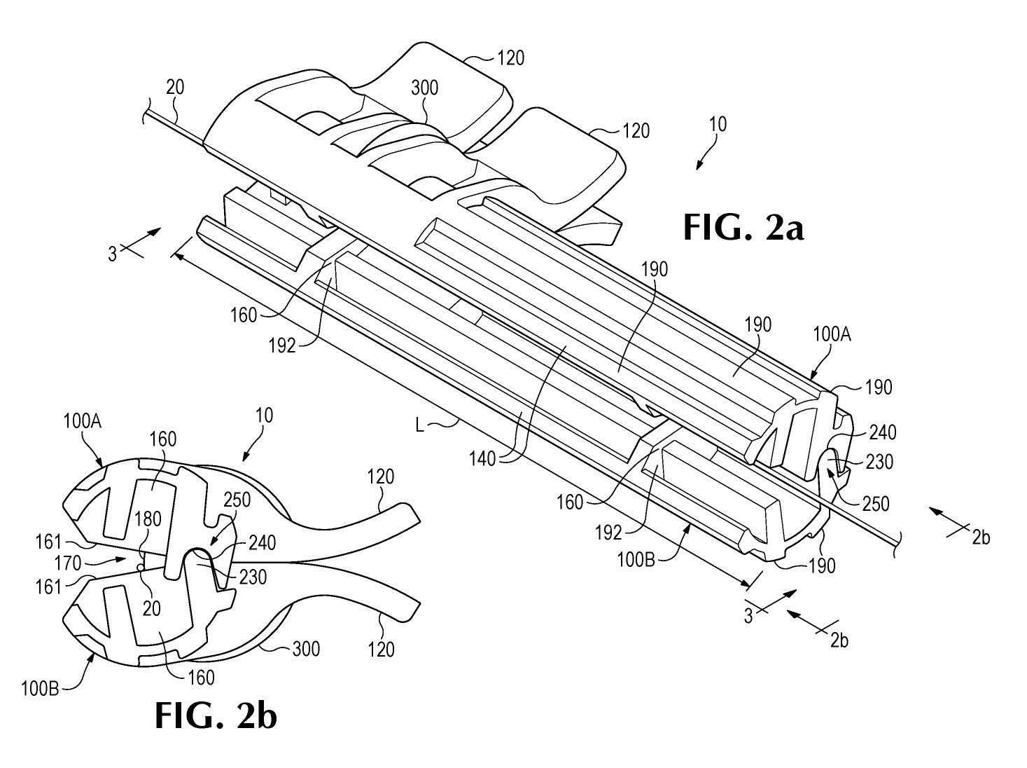

The torque device 10 is shown in FIG. 2a held open, with wire 20 placed inside, to expose the elements on the surfaces of the frames 100A and 100B, which include longitudinal ribs 190 and sectional ribs 192 that include the clamping pads 160. The longitudinal ribs 190 are placed along the longitudinal axis L of the frames 100A and 100B. For convenience and clarity of the drawing parts, the longitudinal axis L is shown outside the device apparatus 10 in FIG. 2a, however, for purposes of this application and in particular for describing the apparatus's elements' movements relative to this axis L, the longitudinal axis L is understood to pass through the longitudinal center line of the assembled torque device, which, in a preferred embodiment is generally occupied by the guidewire 20 during deployment. Pivot bar 230 is shown engaging with pivot socket 240 to form interlocking pivot 250 which extends along a partial or full length of the spinner 140, and alternative embodiments may include pivot 250 elsewhere on the device 10. Also shown are handles 120 and spring 300.

The torque device 10 is shown in the end view of FIG. 2b, taken along lines 2b-2b in FIG. 2a, held open to expose the elements on the frames 100A and 100B, which include clamping pads 160, backstop 180 and clamping surfaces 161 of the clamping pads 160. The guidewire 20 is shown positioned between the open clamping pads 160 and against the backstop 180. Pivot bar 230 is shown engaging with pivot socket 240 to form interlocking pivot 250. Also shown are the handles 120, spring 300 and wire clamp 170 composed of the clamping pads 160 and their included clamping surfaces 161.

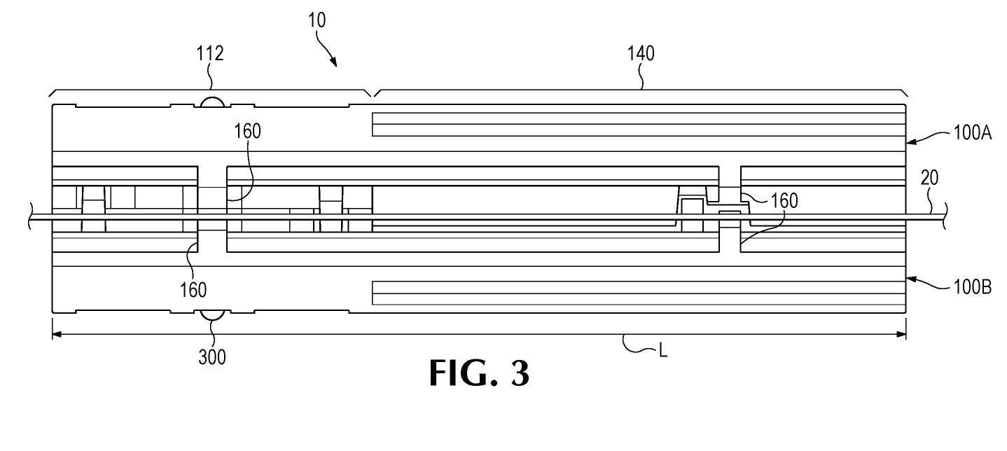

FIG. 3 shows the guidewire 20 and elements of the torque device 10 held open in side view taken along lines 3-3 of FIG. 2a, including clamping pads 160 located in the clamping body 112 and spinner 140, the clamping pads 160 placed perpendicularly to the longitudinal axis L of the device 10. As described above, longitudinal axis L is understood to pass through the longitudinal center line of the assembled torque device 10. The clamping pads 160 on each of the frames 100A and 100B directly face each other in the assembled torque device 10. Also shown is the spring 300.

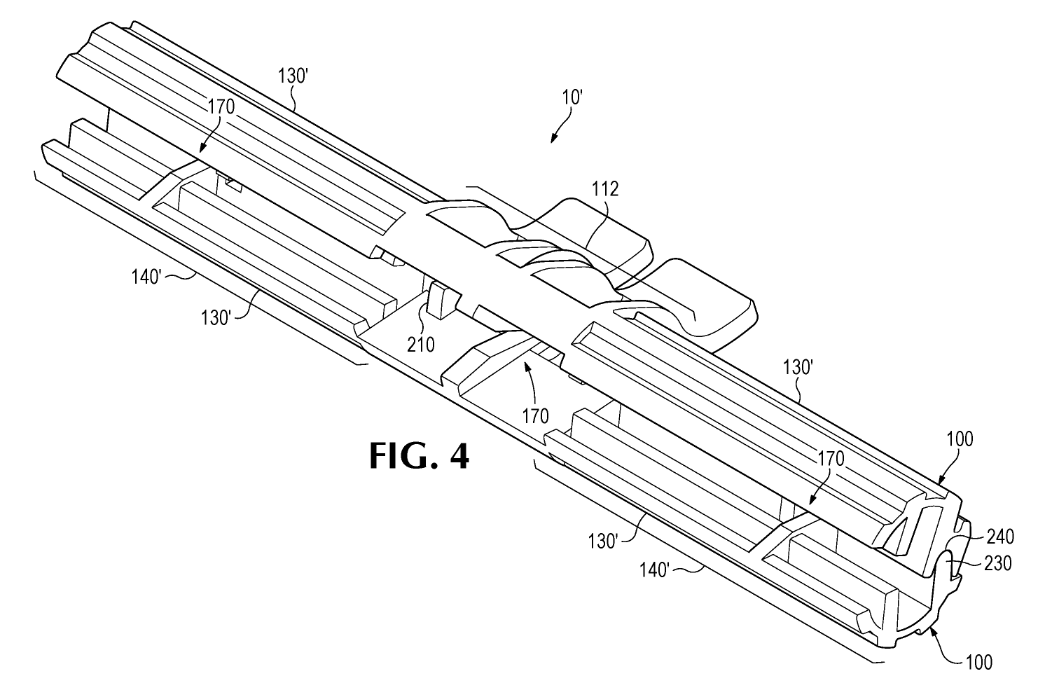

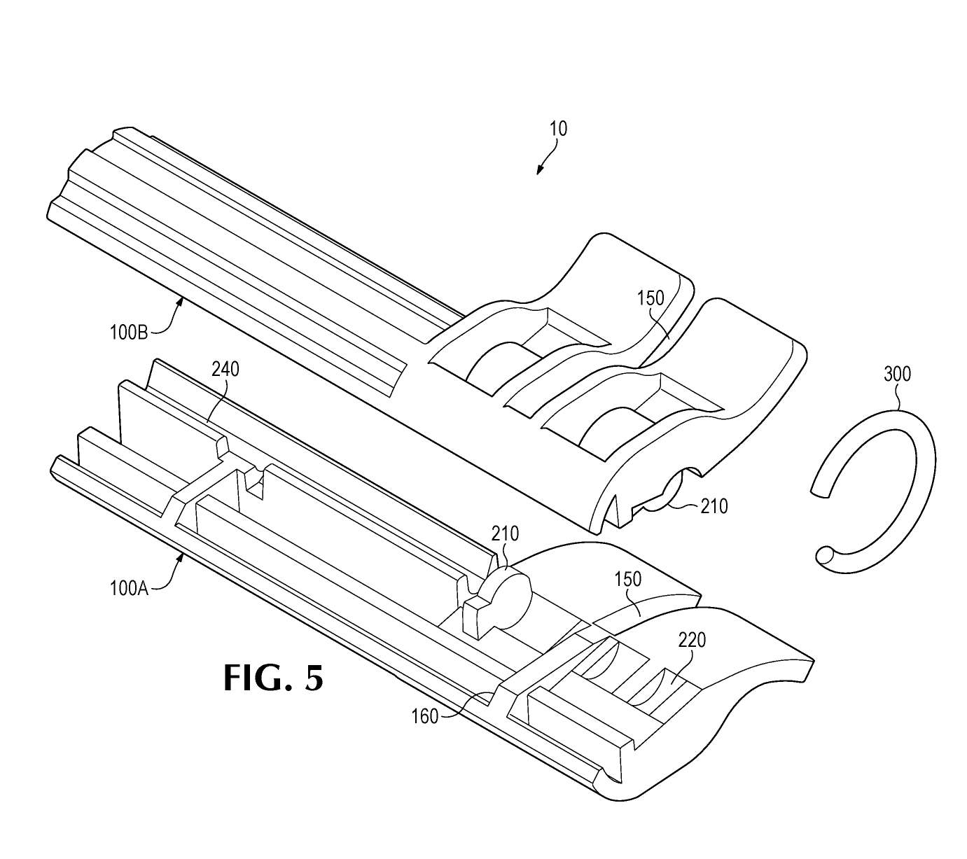

An alternative embodiment, of a bilateral torque device 10′, is shown in FIG. 4. The notable feature differentiating bilateral torque device 10′ from torque device 10 is the inclusion of two spinner handles 130′, instead of only one, on each of the frames 100. The bilateral torque device 10′ thus has two spinners 140′, i.e. a first and a second spinner at opposite ends of the device 10′, instead of only a single spinner 140 as on the device 10. This configuration permits two frames 100 that may be identical, to be used in the assembly of the device 10′, since mirror-imaging of a pivot tab 210, pivot cavity 220 (not shown in this view), pivot bar 230, and pivot socket 240 on the spinner handles 130′. The elements included for the torque device 10 in the descriptions and drawings and elsewhere in this specification are otherwise generally similar to those included in device 10′, for example the wire clamps 170, and clamping body 112.

A spring 300 of the torque device 10 is shown in the exploded view of FIG. 5 as having a “C” shape in a preferred embodiment and when placed in spring slots 150 is used to secure the frames 100A and 100B together and provide clamping force. Also shown are the pivot tabs 210, pivot cavity 220 and elongate pivot socket 240. The spring 300 is placed in spring slots 150 such that its ends are located over at least one wire clamp 170 (not shown in this exploded view) composed of the clamping pads 160. In alternative embodiments, the spring 300 may have different configurations or shapes.

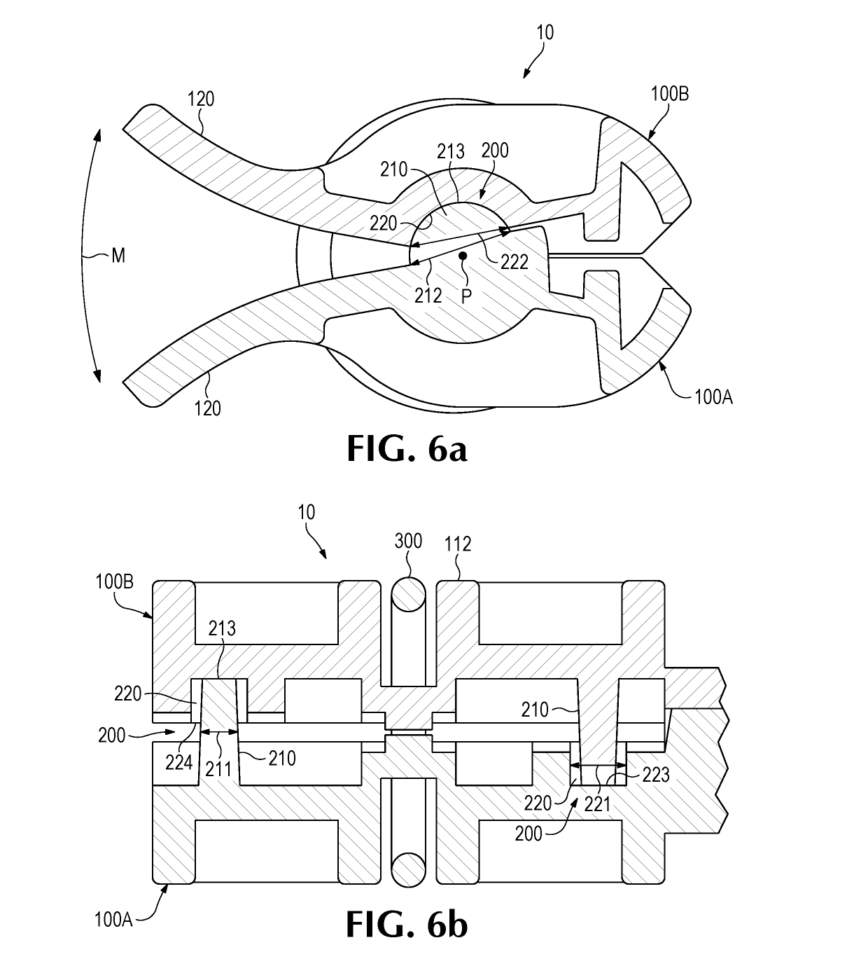

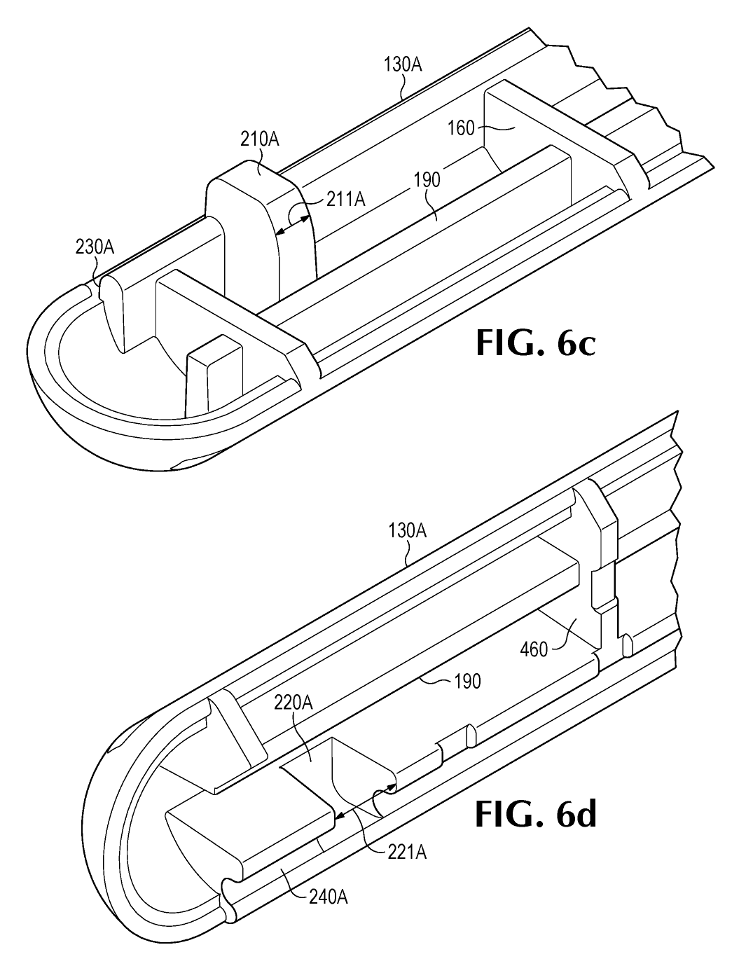

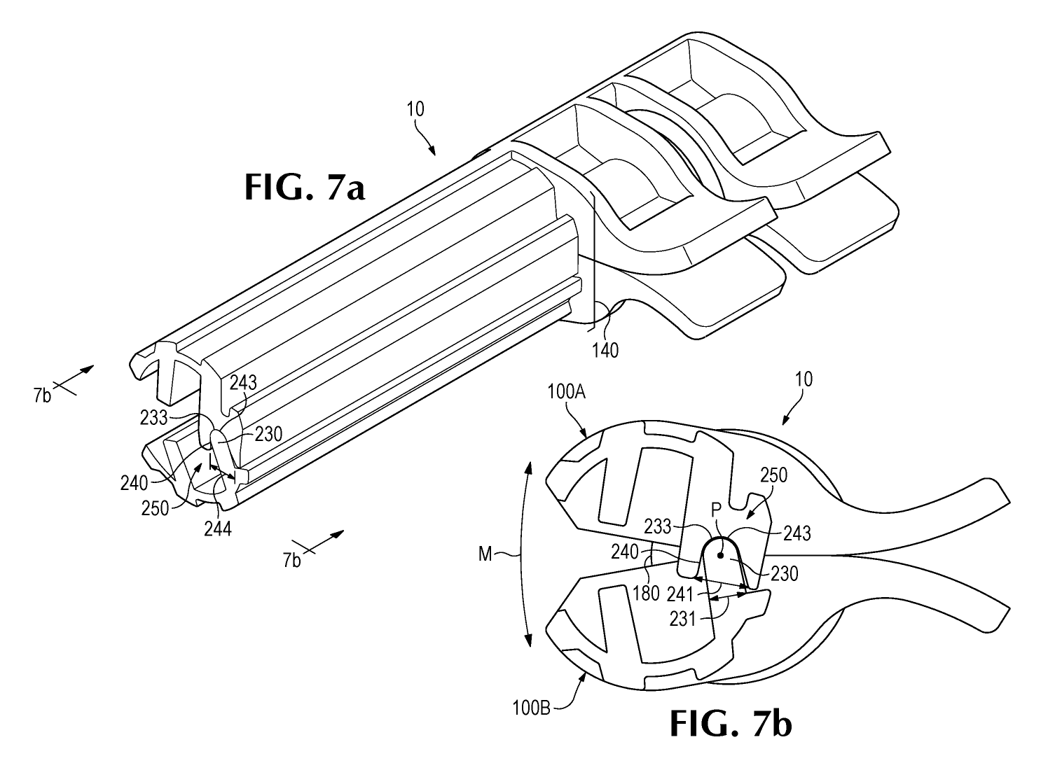

An end partially sectioned view of a centric locking mechanism 200 of the device 10 is shown in FIG. 6a, which includes the pivot tab 210 on frame 100A pivotably mating with the pivot cavity 220 on frame 100B so as to enable pivotable movement M around a pivoting axis P. The pivot tab 210 has a length 212 and a top end 213. The pivot cavity 220 has a length 222, measured at its opening. As mentioned before, longitudinal axis L is understood to pass through the longitudinal center line of the assembled torque device, and may be in a different location than pivoting axis P: axis L and axis P are separate and distinct from each other. Also shown are handles 120.

The centric locking mechanism 200 may be located in the clamping body 112, or, singly or multiply, elsewhere on the device 10, for example in the spinner 140.

In FIG. 6b a side section view of the centric locking mechanism 200 of the assembled device 10 is shown, which includes the pivot tabs 210 on frame 100A pivotably mating with the pivot cavities 220 on frame 100B and held in place with spring 300 in the clamping body 112. The pivot tabs 210 have a width 211 and a top end 213, and the pivot cavity 220 has a width 221, a base 223, and opening 224. Two centric locking mechanisms 200 are shown, though other embodiments may include none, fewer, more or in different locations per unit of the device 10.

The centric locking mechanism 200 is composed of the pivot tab 210 fitting inside of the pivot cavity 220, the cavity's 220 width 221 and length 222 at least slightly exceeding the width 211 and length 212 of the tab 210, the tab 210 fitting through the opening 224 such that the top end 213 of the tab 210 makes contact with the base 223 of the cavity 220. The sides of tab 210 may fit snugly along its length 212 inside the cavity 220, such that pivotable movement M is enabled by movement of the tab 210 inside the cavity 220, but substantial movement in the longitudinal axis L and other movement generally orthogonal to the longitudinal axis L of the frames 100A and 100B relative to each other is restricted or avoided. The top end 213 may not make contact with the base 223 in alternative embodiments.

The shapes and relative dimensions shown in FIGS. 6a and 6b are included in a preferred embodiment but it is within the scope of this invention that different locations, shapes and relative dimensions may alternatively be included, for example, the lengths 212 and 222 and widths 221 and 211 may partially or completely extend to the perimeter of the clamping body 112 or may be located on the spinner 140.

FIGS. 6c and 6d each show an alternative spinner handle 130A, these spinner handles 130A facing each other in an alternative assembled torque device apparatus. FIG. 6c shows a longitudinal rib 190, clamping pad 160, and alternative embodiments of a pivot tab 210A that has a width 211A, and pivot bar 230A. FIG. 6d shows the spinner handle 130A, which faces the handle 130A of FIG. 6c, that includes a longitudinal rib 190, clamping pad 460 (further described herein), and alternative embodiments of a pivot cavity 220A that has a width 221A, and a pivot socket 240A.

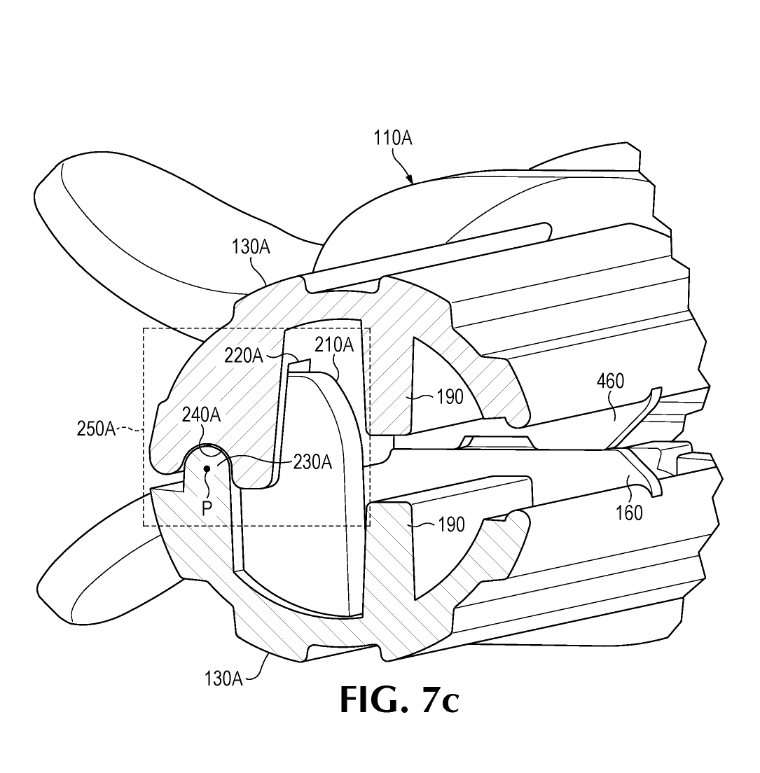

An end perspective view of an interlocking pivot 250 of a torque device 10 is shown in FIG. 7a, which includes the elongate pivot bar 230 pivotably mating with the pivot socket 240. The pivot bar 230 has a length and a top end 233. The pivot socket 240 has a length, a bottom end 243, and an opening 244. The interlocking pivot 250 extends along a partial or full length of the spinner 140, or elsewhere on the device 10.

An end view of the interlocking pivot 250 of torque device 10, taken along lines 7b-7b of FIG. 7a, is shown in FIG. 7b, which includes the pivot bar 230 on frame 100B pivotably mating with the pivot socket 240 on frame 100B so as to enable pivotable movement M around a pivoting axis P. The pivot bar 230 has a width 231 and a top end 233, and the pivot socket 240 has a width 241 and a bottom end 243. Also shown is backstop 180.

The interlocking pivot 250 is composed of the pivot bar 230 fitting inside of the pivot socket 240, the socket's 240 width 241 at least slightly exceeding the width 231 of the bar 230, the bar 230 fitting through the opening 244 such that the top end 233 of the bar 230 makes contact with the bottom end 243 of the cavity 240. The sides of bar 230 may fit snugly inside the socket 240, such that pivotable movement M is enabled by relative movement of the bar 230 inside the socket 240, but generally orthogonal movement relative to the longitudinal axis L (not shown in these FIGS. 7a and 7b), or movement along any axis or plane generally orthogonal to the longitudinal axis L is restricted so as to help prevent the frames 100A and 100B from moving out of parallel relative to each other and relative to the longitudinal axis L. The top end 233 may not make contact with the bottom end 243 in an alternative embodiment.

The shapes and relative dimensions shown in FIGS. 7a and 7b are included in a preferred embodiment but it is within the scope of this invention that different locations, shapes and relative dimensions may alternatively be included, for example, the lengths and widths may differ from what is shown in the figures, or the interlocking pivot 250 may extend partially or completely along the spinner 140 or may be located in the clamping body 112. In an alternative embodiment, the interlocking pivot 250 may be closed at the end of the spinner 140, which FIGS. 7a and 7b show as being open, such that movement along the longitudinal axis L of the pivot bar 230 relative to the pivot socket 240 is restricted or eliminated. Because such movement along the longitudinal axis L is constrained in such and embodiment, the interlocking pivot 250 functions similarly to the centric locking mechanism 200 in that movement in the longitudinal axis L and movement generally orthogonal to the longitudinal axis L of the frames 100A and 100B relative to each other is avoided while permitting pivotable movement M.

In its perspective view with parts removed for clarity, FIG. 7c shows the spinner handles 130A of FIGS. 6c and 6d facing each other in an assembled torque device apparatus, with the pivot bar 230A engaged in the pivot socket 240A and the pivot tab 210A engaged in the pivot cavity 220A to form interlocking key pivot 250A. Also shown are clamping body member 110A, facing clamping pads 160 and 460, and longitudinal ribs 190. To permit engagement that has pivotable movement M (not shown in this view) around pivoting axis P, the width 221A is at least somewhat larger than width 220A (from FIGS. 6c and 6d) and bar 230A engages with socket 240A in the same manner as previously described herein for bar 230 and socket 240, i.e. to form an interlocking pivot 250. As shown in FIG. 7c, the engagement of tab 210A with cavity 220A does not necessarily form a centric locking mechanism 200, and the tab 210A and cavity 220A are shown embedded in the interlocking key pivot 250A, which is formed by bar 230A engaging with socket 240A and the tab 210A engaging with cavity 220A. The tab 210A engaging with cavity 220A does not necessarily enable pivotable movement M around pivoting axis P, this being achieved by the engagement of bar 230A with socket 240A, but instead the tab's 210A insertion into the cavity 220A prevents movement of the spinner handles 130A against each other in opposite directions, i.e. longitudinally or generally orthogonally to or out of parallel with the longitudinal axis L. In a different embodiment, tab 210 and cavity 220 may be substituted for tab 210A and cavity 220A, thereby forming a centric locking mechanism 200, in conjunction with interlocking key pivot 250A.

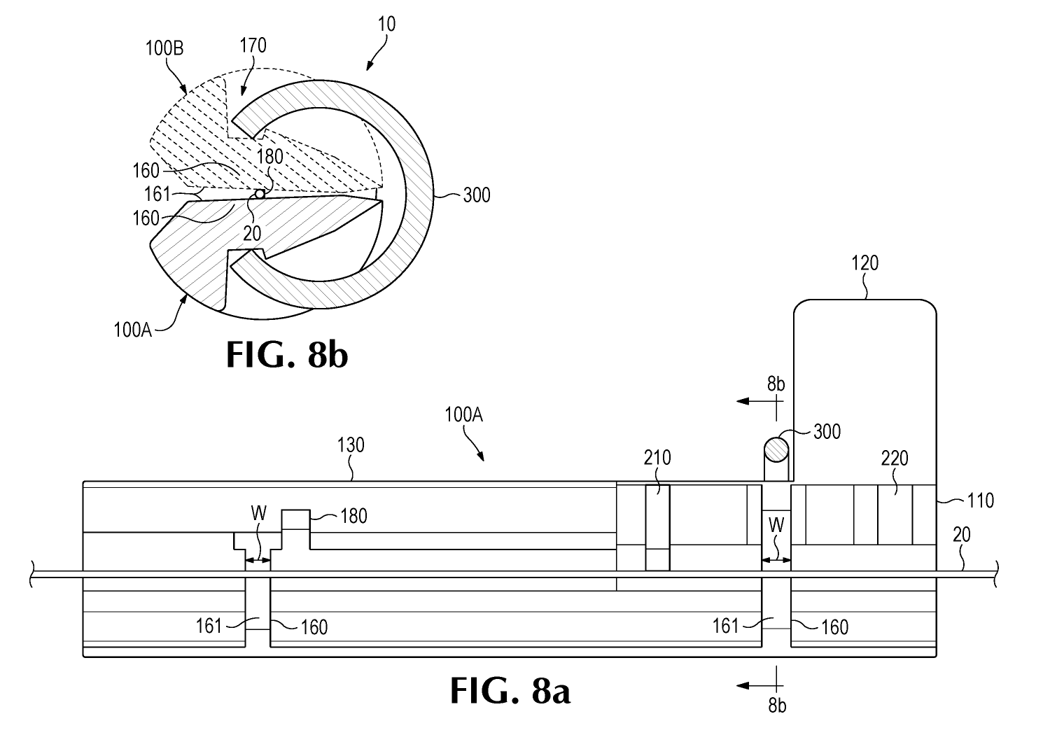

FIG. 8a shows a top view of the interior of another embodiment of first frame 100A. Included are the spring 300 (in section view), backstop 180, and wire clamping pads 160 and their included clamping surfaces 161 in the clamping body member 110 and spinner handle 130, with guidewire 20 placed over the clamping surfaces 161. The clamping surfaces 161 each have a width W that comprises a first dimension of the wire-contacting area of surfaces 161, a second dimension comprising the portion of guidewire 20 in contact with each surface 161 during deployment. Also shown in this embodiment is a single release handle 120 on the first frame 100A, and pivot tab 210 and pivot cavity 220. The widths W of the clamping surfaces 161 may vary from one clamping surface 161 to another within the same device.

FIG. 8b shows an end partially sectioned view of device 10, with parts removed for clarity, taken along lines 8b-8b of FIG. 8a of a guidewire 20 captured in wire clamp 170, which is comprised of the facing clamping pads 160 and included clamping surfaces 161 on frames 100A and 100B. More particularly the guidewire 20 is held in place between the clamping surfaces 161 and against backstop 180, by the clamping force exerted by the spring 300 of the device 10.

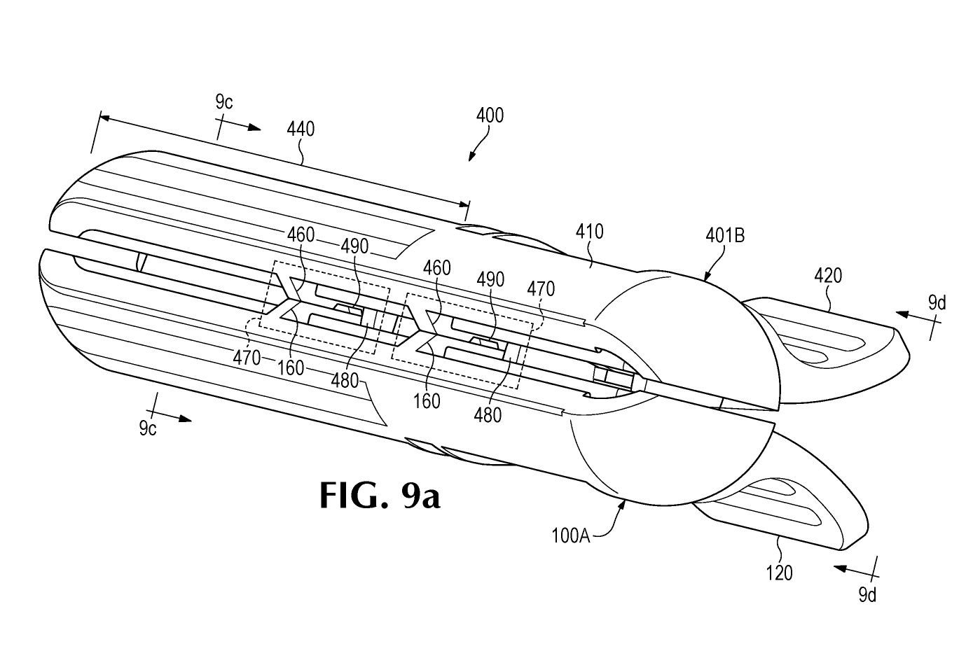

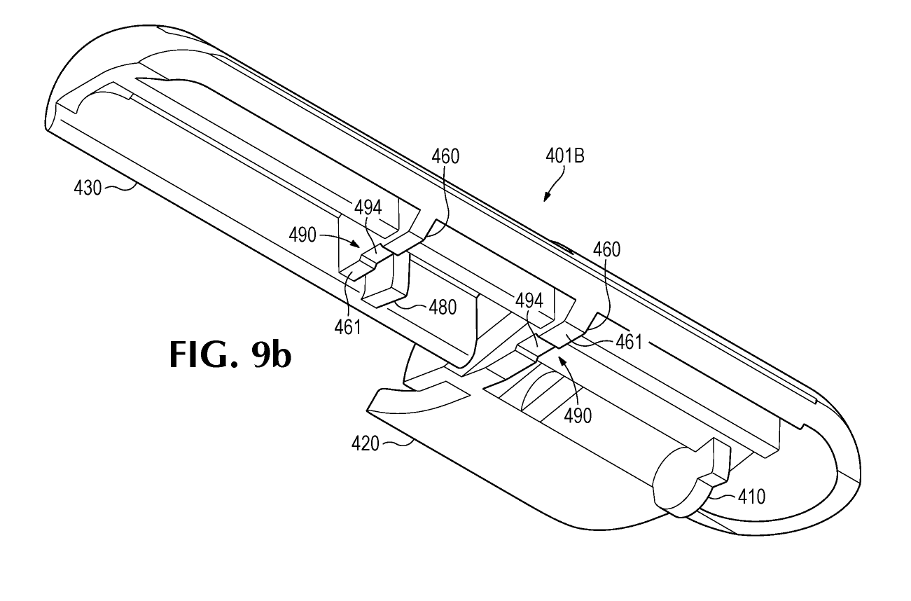

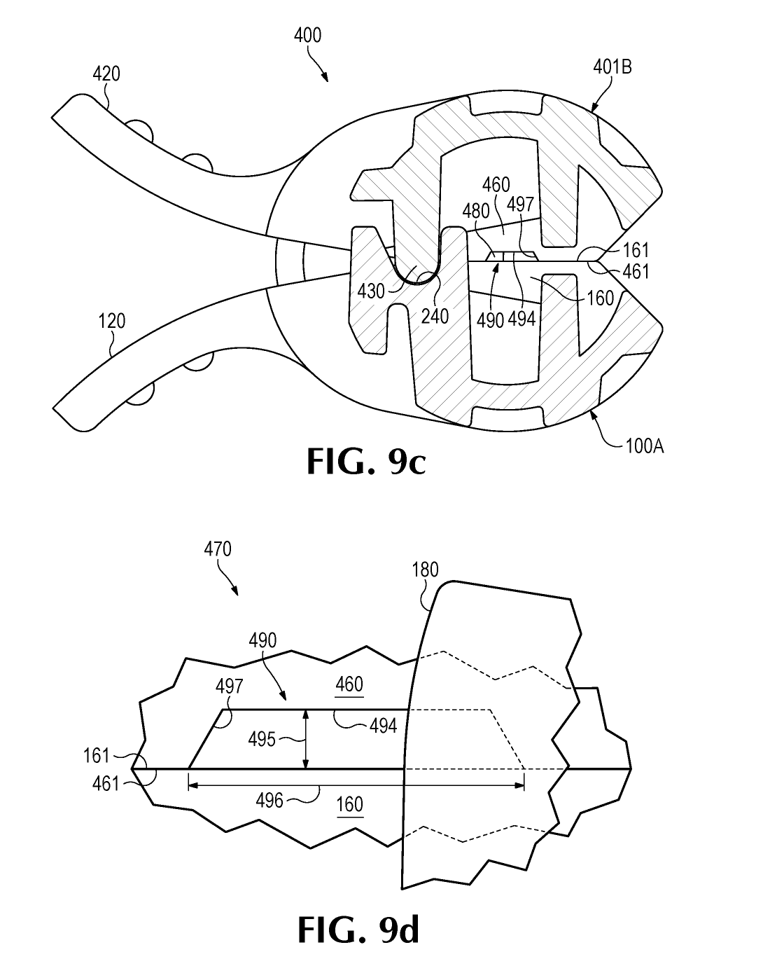

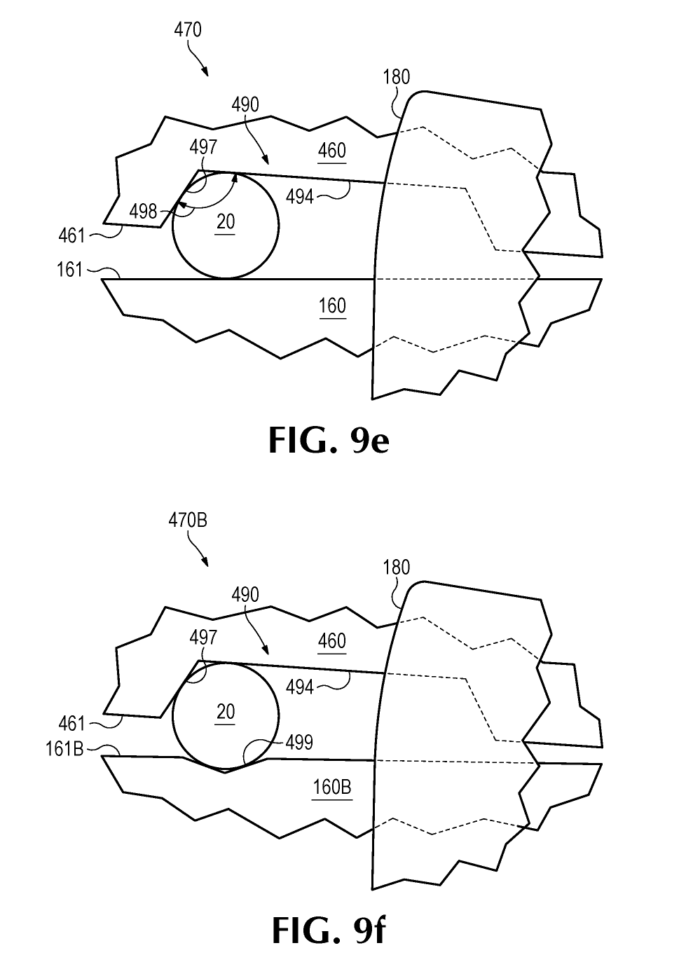

FIG. 9a shows an embodiment of the present invention, i.e. assembled torque device 400, which is identical to the torque device 10 except that it has a second frame 401B instead of second frame 100B. Frame 401B is identical to frame 100B, except that it has wire captivating clamps 470 instead of wire clamps 170. For example, frame 401B includes at least a handle 420, which is a mirror image of the handle 120 on frame 100A, which in a previously described embodiment was a mirror image of frame 100B. Wire captivating clamp 470 includes clamping pad 160 located on frame 100A and clamping pad 460 on frame 401B (and clamping surfaces 161 and 461, not shown in this figure). Frame 401B further includes wire captivating notch 490, as part of wire captivating clamp 470, located on clamping pad 460. Though FIG. 9a shows two such wire captivating notches 490 (as well as facing clamping pads 160) included in the torque device 400, i.e. one inside the spinner 440 and one inside the clamping body member 410, other embodiments may include fewer or more, or in different locations. Wire clamping pad 460, its clamping surface 461 and the wire captivating notch 490 of clamping pad 460, including the wire captivating notch ceiling 494 (not shown in this view), directly face the wire clamping pad 160 and its clamping surface 161. Backstops 480 are also shown in the wire clamps 470.

The bottom perspective view of FIG. 9b shows the interior of second frame 401B. In this view, the pivot tab 410, pivot bar 430 and handle 420 are seen, which are similar to the corresponding parts in frame 100B, previously described (i.e. pivot tab 210, pivot bar 230, and handle 120). Also shown are the backstop 480, clamping pad 460, clamping surface 461, and the wire captivating notch 490, which further includes at least a wire captivating notch ceiling 494 and wire captivating notch retaining edge 497. Two notches 490, one located on the spinner handle 430, are noted in this Figure, though there may alternatively be more or fewer on a frame 401B.

The end view of FIG. 9c, taken along lines 9c-9c of FIG. 9a, shows the frames 100A and 401B of assembled torque device 400 in partially sectioned view, with parts removed for clarity. Shown on Frame 100A are: a handle 120; pivot socket 240; and, clamping pad 160 that further includes clamping surface 161. Shown on Frame 401B are: a handle 420; pivot bar 430, similar to pivot bar 230, which mates with pivot socket 240; backstop 480; clamping pad 460 that further includes clamping surface 461; and, a wire captivating notch 490 that includes a notch ceiling 494, and a notch retaining edge 497.

The end view of FIG. 9d, taken along lines 9d-9d of FIG. 9a, shows the wire captivating clamp 470 in section view, with parts removed for clarity. Clamping pads 160 and 460, with included clamping surfaces 161 and 461, respectively, and backstop 180 are shown. Wire captivating notch 490 includes notch ceiling 494 and notch retaining edge 497. The notch 490 further has a height 495 and a length 496. In this view, the length 496 is greater than the distance between the interior faces of the backstop 180 and retaining edge 497.

The end view of FIG. 9e, taken along lines 9d-9d of FIG. 9a, shows the wire captivating clamp 470 holding a guidewire 20 in section view, with parts removed for clarity. Clamping pads 160 and 460, with included clamping surfaces 161 and 461, respectively, and backstop 180 are shown. Wire captivating notch 490 includes ceiling 494 and retaining edge 497, and an angle 498 that is shown generally to be more than 90 degrees, though it may be more acute or more obtuse in different embodiments of the invention. Though the guidewire 20 is shown to be non-adjacent to the backstop 180, in other embodiments the backstop 180 may be placed such that the guidewire is in contact with said backstop 180 during deployment, instead of or in addition to being in contact with retaining edge 497.

Wire clamps 470 are thus comprised of the facing clamping pads 160 and 460 that each have a clamping surface 161 and 461 respectively, placed on each of the frames 100A and 401B, respectively. The wire-contacting clamping surfaces 161 and 461 of the clamping pads 160 or the wire captivating notch 490, including the retaining edge 497 and ceiling 494, may have a texture or other features for the purpose of improving guidewire 20 securement, for example, by increasing their coefficient of friction without harming such wires 20, in particular coated wires 20. Such textures or other features may include those formed by electrical discharge machining (EDM) textures, grinding, blasting, polishing, milling, etching, lithography, or honing, all of which are well-known to those skilled in the art. No abrasives may be included or placed on the clamping surfaces 161 or 461 or elements of the wire captivating notch 490, since these would damage coated guidewires 20.

The end view of FIG. 9f, taken along lines 9d-9d of FIG. 9a, shows the wire captivating clamp 470B holding a guidewire 20 in section view, with parts removed for clarity. Clamping pads 160B and 460, with included clamping surfaces 161B and 461, respectively, and backstop 180 are shown. Wire captivating notch 490 includes ceiling 494 and retaining edge 497. Clamp 470B is thus similar in structure to clamp 470, except that it additionally includes a wire positioning guide 499 located on clamping surface 161B to assist with guiding the wire 20 into place so that it bears tangentially, i.e. only on a limited portion of the circumference of the wire 20, on surfaces of the ceiling 494, edge 497 and one or more surfaces of the positioning guide 499 for better positioning within the clamp 470B and securement. Though the positioning guide 499 is shown in FIG. 9f to have a “V” shape, it may alternatively have different shapes, angles and dimensions than shown. In the embodiment shown in FIG. 9f, the positioning guide 499 is shown to be concave and having an angle so as to position a wire 20 under the corner formed by edge 497 and ceiling 494. Alternatively, the positioning guide 499 may extend along the entire length of the surface 161B or ceiling 494 or take a shape of a curve, or instead, be convex in shape, for example, an inverted “V” shape, so as to position a wire 20 against the backstop 180 and the ceiling 494. The positioning guide 499 may also be placed in an alternative location, i.e. on the ceiling 494, with the same shapes, angles and dimensions as described for its placement on the clamping surface 161B. As with clamp 470, the wire-contacting clamping surfaces of clamp 470B, including the positioning guide 499, may have a texture or other features for the purpose of improving guidewire 20 securement, for example, by increasing their coefficient of friction.

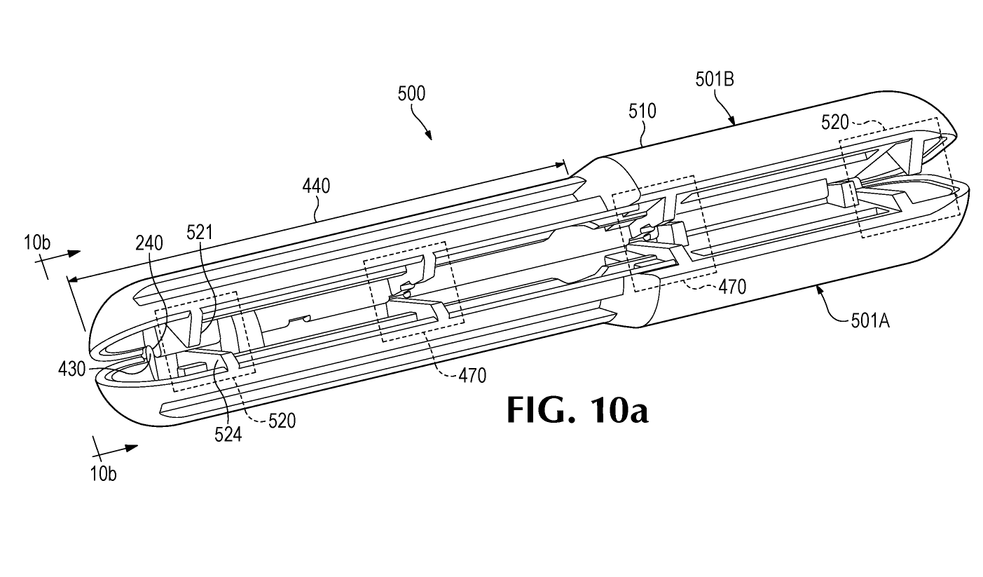

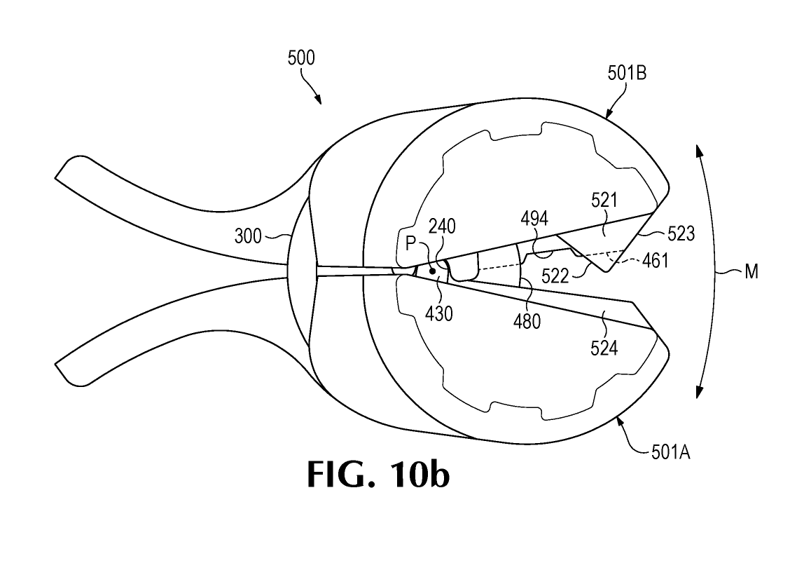

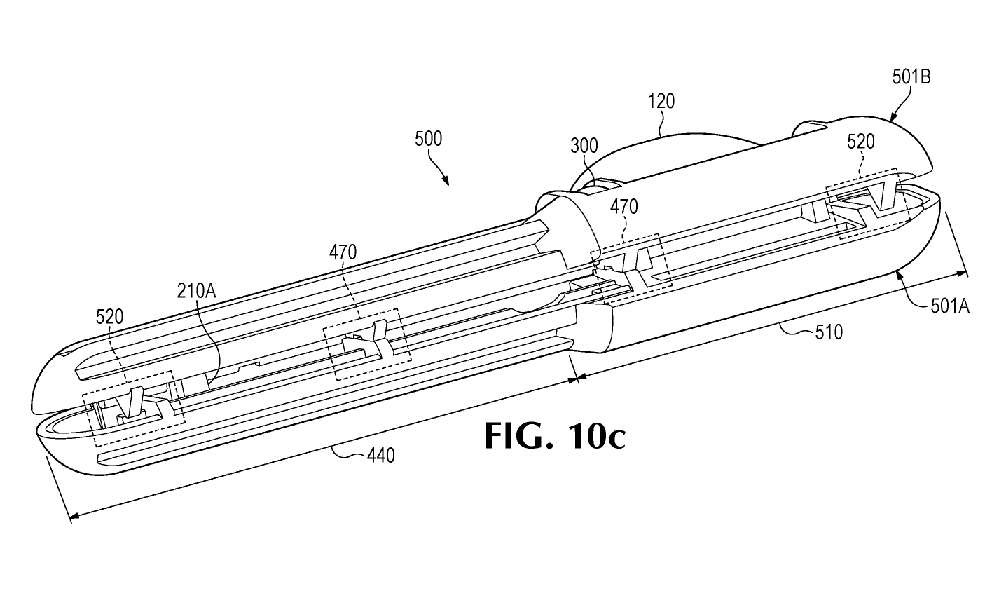

FIG. 10a shows an embodiment of the present invention, i.e. assembled torque device 500, which is identical to the torque device 400 except that it has a first frame 501A and a second frame 501B instead of frames 100A and 401B, respectively. For example, the torque devices 400 and 500 have in common at least the pivot bar 430 and pivot socket 240 and wire captivating clamps 470. The sole difference between the device 400 and the device 500 is the inclusion in the latter of at least one wire catch 520. The wire catch 520, including a wire catch tooth 521 and a wire catch block 524, is placed towards the end of the spinner 440 that is opposite the clamping body 510, although it may also be placed in a different or in additional locations on the device 500, for example between the wire captivating clamps 470, or at the outside end of the clamping body 510. The wire catch tooth 521 bypasses the wire catch block 524 when the device 500 is closed. As shown, the wire catch tooth 521 is located on the outboard side of the wire catch block 524, though these positions may be reversed.

The end view of FIG. 10b, taken along lines 10b-10b in FIG. 10a, shows the torque device 500. Frame 501A includes the pivot bar 430 that further includes pivoting axis P around which the device 500 opens by pivoting action M, the wire catch block 524. Frame 501B includes the pivot socket 240, wire captivating notch ceiling 494 and clamping surface 461, which are parts of the wire captivating clamp 470 shown in FIG. 10a, and the wire catch tooth 521, which further includes an inboard tooth surface 522 and an outboard tooth surface 523, which are also parts of the at least one wire catch 520 shown in FIG. 10a. When the device 500 is closed, a gap is present between the inboard tooth surface 522 of the tooth 521 and the inside surface of the block 524 that faces towards the interior of second frame 501B, said gap capturing the wire 20 to minimize its bending or deflection out of the device's 500 interior during deployment and torquing. An outboard tooth surface 523 is angled inwards to assist in guiding the wire 20 during its insertion into the device 500. No surfaces of the tooth 521 and block 524 make contact with each other in this embodiment. Also shown in this view are backstop 480 and spring 300.

FIG. 10c shows a preferred embodiment of an assembled torque device 500 that includes a spinner 440 and clamping body 510, both formed by a first frame 501A and a second frame 501B held together by spring 300. Also shown in this view are pivot tab 210A, wire catches 520, wire captivating clamps 470 and a single release handle 120 on second frame 501B. Not shown because of the angle of this view are the pivot bar 230A, pivot socket 240A, and pivot cavity 220A, which, together with pivot tab 210A essentially form interlocking key pivot 250A.

The apparatus for a side-loading torque device 10 is used for grasping and manipulating a guidewire 20 during a medical procedure. In one embodiment, the hinging, spring-driven side-loading torque device 10 is composed of a first frame 100A, a second frame 100B, and a spring 300, the frames 100A and 100B each including at least one of: a clamping body member 110, a flat release handle 120, a spinner handle 130, a spring slot 150, or clamping pad 160. The frames 100A and 100B may also further include at least one of: a backstop 180, and a wire entry guide 182. The frames 100A and 100B are joined together by spring 300. The spring 300 joins the frames 100A and 100B together and also provides the clamping force needed for the wire clamps 170 or wire captivating clamps 470 or 470B to secure the guidewire 20 therein during deployment. Further clamping force may be applied by a clinician's fingers while grasping the spinner 140 during manipulation of the device 10. More than one spring 300 may be used thusly in a single torque device 10.

The clamping body member 110 of frame 100A includes a pivot tab 210 and pivot cavity 220 and the spinner handle 130 of frame 100A includes an elongate pivot socket 240. The clamping body member 110 of frame 100B includes a pivot tab 210 and pivot cavity 220 and the spinner handle 130 of frame 100B includes an elongate pivot bar 230. Placement of the pivot tab 210, pivot cavity 220, pivot bar 230 and pivot socket 240 may be alternatively located on the opposite frames, e.g. the pivot bar 230 may be located on frame 100A and the pivot socket 240 may be located on frame 100B. Longitudinal ribs 190 and sectional ribs 192 are placed on the interior surfaces of the frames 100A and 100B to provide structural support and rigidity. The sectional ribs 192 further provide a supporting structure for the clamping pads 160. Longitudinal ribs 190 may also be included on the exterior surfaces of the spinner handles 130 to provide structural support, rigidity, and to enhance a clinician's ability to grip the spinner 140 or 440.

The torque device 10 assembly requires placement of C spring 300 in the spring slot 150 when frames 100A and 100B are placed with their interior surfaces facing each other, the pivot tabs 210 pivotably mating to pivot cavities 220 and pivot bar 230 pivotably mating to pivot socket 240, such registration providing correct positioning and alignment of the frames 100A and 100B relative to each other.

A centric locking mechanism 200 is formed when the pivot tab 210 and pivot cavity 220 pivotably mate together in the assembled devices 10, 10′, 400 or 500; this locking mechanism 200 prevents the frames 100A and 100B from moving against each other longitudinally or along the longitudinal axis L, and enables pivoting action M around a pivoting axis P that further enables the device 10 to open by manually pressing together the flat release handles 120, and the device 10 to close when the release handles 120 are no longer pressed together. More than one centric locking mechanism 200, which enables assembly of the frames 100A and 100B together without a separate axle or pin to hold them together, may be included in a torque device 10, 10′, 400 or 500.

An interlocking pivot 250 is formed when the pivot bar 230 and pivot socket 240 pivotably mate together in the assembled devices 10, 10′, 400 or 500; this pivot 250 enables pivoting action M around a pivoting axis P and prevents the frames 100A and 100B from moving independently off-axis lengthwise, i.e. to prevent them from moving orthogonally to or out of parallel with the longitudinal L axis. Such placements also enable pivoting action M of the frames 100A and 100B around a pivoting axis P that further permits the device 10 to open by manually pressing together the flat release handles 120, and the device 10 to close when the release handles 120 are no longer pressed together. More than one interlocking pivot 250, which enables assembly of the frames 100A and 100B without an axle or pin to hold them together, may be included in a torque device 10, 10′, 400 or 500.

The spring 300 in the assembly of torque devices 10, 10′, 400 and 500 prevents separation of the frames 100, 100A and 100B or 401B, and 501A and 501B. The pivot 250 and interlocking key pivot 250A further enables the device 10 to open by manually pressing together the flat release handles 120, and the device 10, 10′, 400 or 500 to close when the release handles 120 are no longer pressed together.

The centric locking mechanism 200, interlocking pivot 250 and interlocking key pivot 250A are the only members of a group defined herein as pivoting assembly elements, i.e. they can each be termed more generally as a pivoting assembly element.

The centric locking mechanism 200, interlocking pivot 250 and interlocking key pivot 250A enable the frames 100, 100A and 100B or 401B, and 501A and 501B to be secured together when the spring 300 is inserted into the spring slots 150 without requiring that a separate pin or axle be inserted through axle holes or pin holes in the frames 100, 100A and 100B or 401B, and 501A and 501B. Further, when the spring 300 is placed in the spring slots 150 of the assembled device 10, 10′, 400 or 500 and the pivot bar 230 is placed in the pivot socket 240 in an assembled device 10, the frames 100, 100A and 100B or 401B, and 501A and 501B are prevented from moving relative to each other in the longitudinal axis L since the spring 300 limits travel of the frames by pressing against the interior walls of the spring slot 150. Spring placement in a device 10, 10′, 400 or 500, which includes engagement of the bar 230A with the socket 240A in an assembled device 10, 10′, 400 or 500, functions similarly. Assembled torque devices 10, 10′, 400 and 500 as shown in this application include the spring 300 and both the centric locking mechanism 200 and interlocking pivot 250. Alternative embodiments may include only one or a combination of the pivoting assembly elements, more particularly the locking mechanism 200, pivot 250, or key pivot 250A. The centric locking mechanism 200, interlocking pivot 250 and interlocking key pivot 250A obviate the requirement for a pin or axle and their corresponding holes in the frames 100, 100A and 100B or 401B, and 501A and 501B with which to join the frames 100A and 100B or 401B, and 501A and 501B together. Thus, in each of the pivoting assembly elements, only the spring 300 joins together frames 100, 100A and 100B or 401B, and 501A and 501B, and the torque devices 10, 10′, 400 and 500 exclude axles, pins or living hinges as elements for joining or otherwise connecting together said frames 100, 100A and 100B or 401B, and 501A and 501B.

In the assembled torque device 10, the clamping pads 160 in the clamping body members 110 or spinner handles 130 of frames 100A and 100B directly face each other. Backstops 180, located near the clamping pads 160 and towards the closed side of the device 10, help prevent accidental misplacement of the guidewire 20 to a location off of the clamping pads 160 and help with proper placement in a direction generally in the longitudinal axis L.

The C spring 300 is located in the spring slots 150 of the frames 100A and 100B when their interior surfaces face each other and not only provides a clamping force with which to securely grasp a guidewire 20, but also to secure the frames 100A and 100B together by preventing their separation, and in conjunction with the pivoting assembly elements, preventing movement in directions orthogonal to the longitudinal axis L. The ends of the spring 300 are disposed in the spring slots 150, which may be positioned directly over at least one wire clamp 170 so as to maximize the spring's 300 clamping force that is directed onto the guidewire 20. One or more additional wire clamps 170 may be placed elsewhere in the device 10, more particularly at a location inside the spinner 140 or the clamping body 110.

A wire clamp 170 of the assembled device 10 is formed by the clamping surfaces 161 of the facing clamping pads 160 together with the force exerted by the spring 300. Further clamping force may be applied by a clinician's fingers while grasping the spinner 140 during manipulation of the device 10. When the assembled device 10 is in operation and the facing clamping surfaces 161 clamp the guidewire 20, longitudinal and rotational movement of the guidewire 20 relative to the assembled device 10 is prevented when clamping force is applied. The device 10 excludes clamping surfaces that are interleaved or alternating, i.e. surfaces that do not directly face each other. In alternative embodiments, the wire clamp 170 may be placed in one or more locations in the clamping body 112 or spinner 140.

The width W of the clamping surfaces 161, is limited compared to the prior art, thus resulting in a smaller wire 20-contacting area, thus they may more particularly be termed limited-width clamping surfaces 161. Each surface 161 has a wire-contacting area that is determined by its width W and the portion of the guidewire 20 in contact with the surface 161. This limitation, i.e. the relatively limited wire-contacting area, as described elsewhere herein is for the purpose of maximizing normal force and local pressure directed onto the guidewire with a given spring 300. The clamping pad 160 width and limited-width clamping pad surface 161 width is in the range of 0.025″ to 0.300″, more particularly in the range of 0.050″ to 0.250″. The surface width may vary depending on the materials used in the clamping pads 160, the textures applied to the clamping surfaces 161, the force applied by the spring 300 and the numbers of wire clamps 170 in each device 10. An embodiment of the device 10 may include a total wire-contacting area, i.e. the sum of the wire-contacting areas of all the wire clamps in a single unit of the device 10, that is in the range of 0.025″ to 1.0″, more particularly in the range of 0.025″ to 0.500″. The wire captivating notch 490, clamping surface 461, wire captivating notch retaining edge 497, wire captivating notch ceiling 494, and wire positioning guide 499 have surface widths W generally the same as the limited-width clamping surface 161 widths. Thus, they may more particularly be termed: limited-width wire captivating notch 490, limited-width clamping surface 461, limited-width wire captivating notch retaining edge 497, limited-width wire captivating notch ceiling 494 and limited-width wire positioning guide 499. In other embodiments, such widths may be larger or smaller than the clamping pad's 161 width, but where they are larger, they are not termed limited-width. For clarity and for purposes of interpreting the Claims presented in this application, the term limited-width is applied to elements of the wire clamps 170, 470, and 470B as described in this paragraph, that have widths in the range of 0.025″ to 0.300″, more particularly in the range of 0.050″ to 0.250″.

A spinner 140 is composed of the facing spinner handles 130 or 130A that, when the device 10 is closed or is clamping a guidewire 20, form a barrel having a generally circular cross-section that extends from the interior end of the clamping body 112 and has a length 141 through which the guidewire 20 passes. The length 141 and width 142 are sufficient for a clinician to grasp and manipulate the spinner 140 with at least two fingers, for example an index finger and thumb. In an alternative embodiment, the bilateral torque device 10′ may include at least two spinners 140′, each at opposite ends of the clamping body 112. The spinners 140′ on a device 10′ are shown to be similar in FIG. 4 but they may have dimensions and components that are different, for example, one of the spinners 140′ may have a spinner handle length 141, or a wire clamping pad 470 that has different dimensions than a wire clamping pad 470 on the opposite spinner 140′.

The assembled torque device 400 (as shown in FIGS. 9a-9f) is identical in structures, functions and operation to the torque device 10 except that it has a second frame 401B instead of second frame 100B. Frame 401B is identical in structures and functions to frame 100B, except that it has a wire captivating clamp 470 instead of a wire clamp 170.

The wire captivating clamp 470 is comprised of the directly facing wire clamping pads 160 and 460 and their included clamping surfaces 161 and 461, respectively, and the wire captivating notch 490 that further includes the wire captivating notch ceiling 494 and wire captivating notch retaining edge 497. The backstop 180 physically prevents a guidewire 20 from moving closer towards the pivoting axis P (as shown in FIG. 7b) and the closed side of the device 400. A different embodiment of the wire captivating clamp 470 may further include a wire positioning guide 499 that helps to position a wire 20 within the notch 490 either under the retaining edge 497 or against the backstop 180 or 480. The wire-contacting clamping surfaces 161 and 461, wire captivating notch ceiling 494, wire captivating notch retaining edge 497, or positioning guide 499 may further include a texture or other features for the purpose of increasing their coefficient of friction, or for better guidewire 20 securement.

Further, in the assembled torque device 400, a gap may be disposed between the facing clamping surfaces 161 and 461 when a guidewire 20 is held between the surfaces of the captivating notch 490 and clamping surface 161. The notch 490 further provides a mechanical barrier to help prevent the guidewire 20 from being accidentally dislodged from the torque device 400: in addition to the application of clamping force and the friction between the clamping surface 161, ceiling 494, and the inserted guidewire 20, the retaining edge 497 also applies clamping force and physically interferes with lateral movement of the guidewire 20 away from pivoting axis P or the closed side that results from a force bearing upon it from the direction opposite the retaining edge 497, which could otherwise cause the guidewire's 20 dislodgement or migration towards the opening side of the torque device 400. Such lateral movement could be caused by careless handling by an operator, or by dropping or bumping or otherwise disturbing torque device 400 with a retained guidewire 20. As seen in the Figures, clamping surface 161 has a total length greater than clamping surface 461, the amount of this total length difference generally comprising the length 496 of the wire captivating notch 490.

The height 495 is generally less than the minimum diameter of a guidewire 20 that is intended to be inserted into and retained inside the torque device 400. For example, the height 495 of notch 490 in a torque device 400 to be used with a guidewire 20 having a diameter of 0.018 inches would be generally in the range of 0.001 inches to 0.01799 inches, more particularly in the range of 0.002 inches to 0.015 inches. Thus, during deployment with a guidewire 20 retained in the clamp 470, the clamping surfaces 161 and 461 are separated, as seen in FIGS. 9e and 9f. In another alternative embodiment, the height 495 may be greater than the maximum diameter of a guidewire 20 that is intended to be inserted into the torque device 400. The angle 498 between the captivating edge 497 and ceiling 494 may be equal to or more or less than 90°, but may be more than 90° in a preferred embodiment.

The length 496 is generally equal to the maximum diameter of a guidewire 20 that is intended to be inserted into and retained inside the torque device 400 but may be larger or smaller. For example, the length 496 of notch 490 in a torque device 400 to be used with guidewires 20 having a diameter of 0.018 inches would be generally in the range of 0.010 inches to 0.100 inches, more particularly in the range of 0.018 inches to 0.05 inches. In another embodiment, the length 496 may be less than the minimum diameter of a guidewire 20 that is intended to be inserted into the torque device 400. Further, where more than one wire captivating clamp 470 is included in a torque device 400, each clamp 470 may have a height 495 or length 496 that is different from the other clamps 470. For example, a torque device 400 may include three wire captivating clamps 470, one of which has a height 495 and length 496 that are larger than in the other two clamps 470. A single device 400 may have at least one captivating clamp 470 able to accommodate multiple sizes of wires 20 as described herein, e.g. wires 20 including those generally having the diameters (in inches) of 0.010, 0.014, 0.018, and 0.035. Some guidewires 20 have specific widths different than those explicitly described herein and these are also intended to be used with the present invention.

The wire captivating clamp 470 is shown to have a single notch 490 located on a single frame, in this instance frame 401B; thus, there is only a single ceiling 494 and a single retaining edge 497 in clamp 470. Another embodiment of a clamp 470 may have directly facing notches 490: instead of clamping surface 161 facing ceiling 494 as presently shown in torque device 400, each of two clamping pads 460, one on each frame of an assembled device 400, would include a notch 490 so that ceilings 494 directly face each other, thereby forming a dual-notch wire captivating clamp. The height 495 would be measured from ceiling 494 to ceiling 494. There would thus be two retaining edges 497, one on each clamping pad 460. The two retaining edges 497 may be of either equal or unequal height 495. Further, assembled torque device 400 as shown in this application shows two wire captivating clamps 470 in the torque device 400. Alternative embodiments may include one or more wire clamps 170 and one or more wire captivating clamps 470, i.e. only one, or the other, or both of these clamps 170 and 470 together included in a single device. In the wire captivating clamp 470, the wire clamping pad 160 and its included clamping surface 161 directly face the wire clamping pad 460, its included clamping surface 461 and its included wire captivating notch ceiling 494. In some embodiments, the wire captivating clamp 470 may have at least one clamping surface 161 or wire captivating notch 490 that are limited-width. Clamping pads, either 161 or 461 directly face the clamping pads, either 161 or 461 on the facing frame.

The ceiling 494 as presented herein, e.g. in FIG. 9d, is shown to be flat and parallel to the facing clamping surface 161. Alternative embodiments may include a wire captivating notch 490 that has a ceiling 494 that is not flat, or which has a surface that generally conforms to the circumference of the guidewire 20 that is intended to inserted into the device, or which is not parallel to the facing clamping surface 161. Alternative embodiments may also include notches 490 or clamping surfaces 161 that are formed from a material different than that used in the frames 160 or 460, for example, a thermoplastic elastomer or a natural rubber or other non-rigid material.

The wire catch 520 of the torque device 500 includes a wire catch block 524 and wire catch tooth 521 that further includes an inboard tooth surface 522 and an outboard tooth surface 523. FIGS. 10a and 10b show the wire catch 520 placed at the end of the device 500 opposite the clamping body 510, though it may be included in other or in additional locations, for example at the clamping body 510 end or between the wire captivating clamps 470. Whereas the wire clamps 170 and wire captivating clamps 470 are intended to securely grasp onto and prevent both rotational and longitudinal movement of the wire 20 relative to the clamps 170 or 470, the wire catch 520 is intended to restrict movement of the wire 20 while it is secured by clamps 170 and/or 470, without securely grasping said wire 20. When the device 500 is held open, a wire 20 is able to pass through the space between the tip of the wire tooth 521 and the top surface of the wire catch block 524. As seen in FIG. 10a, the tooth 521 on frame 501B and block 524 on frame 501A and are offset so that when the device 500 is closed, the tooth 521 and block 524 bypass each other and are generally adjacent though not necessarily in contact when the device 500 is closed.

Because guidewires 20 are generally flexible to varying extent, they may bend during deployment. The wire captivating clamps 470, if located towards the center of the device 500, could allow such bending such that the wire 20 may deflect through the opening between the edges on the side of the frames 501A and 501B, which may be undesirable when the operator is deploying the wire 20 during a procedure. The wire catch 520, though it does not necessarily make contact with the wire 20 at all times, serves to loosely restrain the wire's 20 movement and prevent its deflection out of the side of the device 500, for example while an operator torques or longitudinally moves said wire 20 during deployment in a patient. Contact of the wire 20 with the wire catch 520 would occur only during its deflection and only with the inboard tooth surface 522 or top surface of wire block 524. Clamping force is not applied to the wire 20 by the wire catch 520.

When the device 500 is held open a wire 20 may be passed through the side and past the tip of the wire catch tooth 521. Once the device 500 is allowed to close, with the clamps 170 or 470 securely grasping the wire 20, the tip of tooth 521 travels past the top surface of wire block 524, closing access to the outboard area of the device 500 by the wire 20, while leaving ample space for the wire 20 move freely in the space bounded by the wire catch 520 and wire block 524, and interior surfaces of the spinner 440.

The inboard tooth surface 523 is angled so as to help guide the wire 20 inward once it has been inserted through the side and towards the center of the device 500, as it is closed. The outboard tooth surface 524 is angled so as to help guide the wire 20 into the device 500 while it is held open during placement of the wire. Angles other than those shown in FIGS. 10a and 10b may be used for the surfaces 523 and 524, or angles on one or both surfaces 523 and 524 may be absent.

The hinging, spring-driven, side-loading guidewire torque device apparatuses (in the interest of brevity and reader convenience, the term “torque device apparatuses” shall hereinafter refer to torque devices, 10, 10′, 400 and 500, except where specifically indicated otherwise) are used to manipulate a guidewire 20 used by a clinician in a medical procedure for the purposes of: i) loading a guidewire 20 from the side instead of threading it though an end and securely retaining such guidewire 20 within the torque device apparatuses; and ii) advancing and rotating such guidewire 20 during a medical procedure. The torque device apparatuses are each assembled from two frames, 100, 100A and 100B, or 100A and 401B, or 501B and 501A, respectively, joined together only by one or more springs 300. Said torque device apparatuses include: at least one of a centric locking mechanism 200, an interlocking pivot 250, and an interlocking key pivot 250A; at least one of: a spinner 140 or 440, release handles 120 or 420, wire clamp 170, and wire captivating clamp 470. The torque device apparatuses exclude axles, pins or living hinges for joining or otherwise connecting said frames 100, 100A and 100B, or 100A and 401B, or 501A and 501B, together.

A hinging securement means of the assembled torque device apparatuses is used for the purposes of: i) positioning for assembly the frames 100, 100A, 100B, 401B, 501A or 501B and then joining them together with one or more springs 300, ii) enabling only pivotable movement M of one of such frames 100, 100A, 100B, 401B, 501A or 501B against the other about a pivoting axis P, and preventing significant movement of one against the other in the longitudinal axis L or movement in an axis generally orthogonal to the longitudinal axis L. Said hinging securement means is defined as either: i) all of the centric locking mechanisms 200 in a single unit of the assembled torque device apparatuses if interlocking pivots 250 and interlocking key pivots 250A are not present; or ii) all of the interlocking pivots 250 in a single unit of the assembled torque device apparatuses if centric locking mechanisms 200 and interlocking key pivots 250A are not present; or iii) all of the interlocking key pivots 250A in a single unit of the assembled torque device apparatuses if other pivoting assembly elements are not present; wherein the assembled torque device apparatuses include at least one spring 300 placed between the interior walls of the spring slots 150. More simply, the hinging securement means comprises any combination of one or more centric locking mechanisms 200, interlocking pivots 250, or interlocking key pivots 250A in a single unit of the assembled torque device apparatuses, and is for the purposes of enabling positioning and assembly of the frames 100A, 100B, 401B, 501A or 501B of a side-loading torque device apparatus (i.e. 10, 10′, 400 or 500), and for enabling and permitting only pivotable movement M of one frame against the other around a pivoting axis P. The hinging securement means shall be construed only as understood herein and restricted to the apparatus and functions described, in their entirety, in this Specification and the Figures.

A pivoting means of the assembled torque device apparatus is used for the purposes of: i) positioning and assembling frames 100A, 100B, 401B, 501A or 501B together with at least one spring 300; and, ii) enabling pivotable movement M about a pivoting axis P of the pivot bar 230 or 230A and pivot socket 240 or 240A; and iii) preventing lateral movement of one frame 100A, 100B, 401B, 501A or 501B against the other or movement generally orthogonal to the longitudinal axis L, and preventing the frames 100A, 100B, 401B, 501A or 501B from moving independently off-axis lengthwise, i.e. to prevent them from moving out of parallel with the longitudinal axis L. Said pivoting means is defined as either i) all of the centric locking mechanisms 200 in a single unit of the assembled torque device apparatuses if interlocking pivots 250 and interlocking key pivots 250A are not present; or ii) all of the interlocking pivots 250 in a single unit of the assembled torque device apparatuses if centric locking mechanisms 200 and interlocking key pivots 250A are not present; or iii) all of the interlocking key pivots 250A in a single unit of the assembled torque device apparatuses if other pivoting assembly elements are not present; wherein the assembled torque device apparatus (i.e. 10, 10′, 400 or 500) includes at least one spring 300 placed in the spring slots 150. More simply, the pivoting means comprises any combination of one or more centric locking mechanisms 200, interlocking pivots 250, or interlocking key pivots 250A in a single unit of the assembled torque device apparatuses. The pivoting means shall be construed only as understood herein and restricted to the apparatus and functions described, in their entirety, in this Specification and the Figures.

A device securement means of the assembled torque device apparatus is used for the purposes of: i) positioning and assembling frames 100A, 100B, 401B, 501A or 501B together with at least one spring 300 and without living hinge, axle or pin or pinhole to join them together; and, ii) enabling pivotable movement M about a pivoting axis P; and iii) preventing longitudinal (i.e. along the longitudinal axis L) movement of one frame 100A, 100B, 401B, 501A or 501B against the other or movement generally orthogonal to the longitudinal axis L, and preventing the frames 100A, 100B, 401B, 501A or 501B from moving independently off-axis lengthwise, i.e. to prevent them from moving out of parallel with the longitudinal axis L. Said device securement means is defined as either: i) all of the centric locking mechanisms 200 in a single unit of the assembled torque device apparatuses if interlocking key pivots 250A are not present; or ii) all of the interlocking key pivots 250A in a single unit of the assembled torque device apparatuses if centric locking mechanisms 200 are not present; or iii) all of the centric locking mechanisms 200 and interlocking key pivots 250A in a single unit of the assembled torque device apparatuses; wherein the assembled torque device apparatus (i.e. 10, 10′, 400 or 500) includes at least one spring 300. The device securement means shall be construed only as understood herein and restricted to the apparatus and functions described, in their entirety, in this Specification and the Figures.

A clamping means of the assembled torque device apparatuses, used for the purpose of securely grasping the guidewire 20 so as to avoid its rotational and longitudinal movement inside of and relative to the torque device apparatus (i.e. 10, 10′, 400 or 500), is exclusively defined as all of the wire clamps 170 or 470 or 470B in a single unit of the assembled torque device apparatuses that further includes at least one spring 300 placed in the spring slots 150. More simply, the clamping means is for the purpose of securely grasping a guidewire 20 so as to avoid its rotational and longitudinal movement inside of and relative to said torque device apparatuses. The wire clamp 170 of the clamping means includes at least the clamping pads 160, their included clamping surfaces 161. The wire clamp 470 of the clamping means includes at least a clamping pad 160 and its included clamping surface 161, a clamping pad 460 and its included clamping surface 461, wire captivating notch 490 and its included ceiling 494 and retaining edge 497, the notch 490 having a height 495 and length 496. Another embodiment of a clamping means, clamp 470B in particular, may further include a wire positioning guide 499. One or more elements of the clamps 170, 470 or 470B in a preferred embodiment, may be of limited-width. The clamping means shall be construed only as understood herein and restricted to the apparatus and functions described, in their entirety, in this application, and as shown in the Figures.

A guidewire manipulation means of the assembled torque device apparatuses, used by an operator to grasp with at least two fingers, for example the thumb and index finger, the assembled torque device apparatus (i.e. 10, 10′, 400, or 500) with guidewire 20 secured inside and to move such apparatus so as to rotate, and advance and withdraw the wire 20 during deployment in a patient, is exclusively defined as any combination of one or more of the spinners 140 and 440, and wire clamps 170 and wire captivating clamps 470 and 470B in a single unit of the assembled device apparatus, that further includes at least one spring 300 placed in the spring slots 150. In a preferred embodiment of the guidewire manipulation means, one or all elements of the clamps 170, 470 or 470B may be of limited-width. More simply, the guidewire manipulation means is for the purpose of enabling an operator to grasp the torque device apparatus, and to manipulate it so as to rotate, and advance and withdraw the guidewire 20 retained therein during its deployment in a patient. The guidewire manipulation means shall be construed only as understood herein and restricted to the apparatus and functions described, in their entirety, in this Specification and the Figures.

The hinging securement means, pivoting means and clamping means exclude axles, pins and living hinges as elements for connecting components of the assembled torque device apparatus (i.e. 10, 10′, 400 and 500), which may more particularly be described as a hinging, spring-driven, side-loading guidewire torque device apparatuses. Therefore, generally, the hinging, spring-driven, side-loading guidewire torque device apparatus is defined for purposes of the present application as a device for manipulating a guidewire used by a clinician in a medical procedure, that is assembled from two frames and at least one spring without including or using axles, pins or living hinges as elements for connecting said frames, and that further permits: i) loading of a guidewire from the side instead of threading it though an end; ii) securing such guidewire within the device to prevent slippage during rotation and other movement of the torque device apparatus; and iii) advancing and rotating such guidewire during a medical procedure.

A wire captivating means of the assembled torque device 400 or 500 is used during deployment, i.e. while retaining a guidewire 20, for the purpose of physically interfering with unintentional lateral, i.e. side-to-side (perpendicular to the longitudinal axis L), movement of the guidewire 20 caused by a force applied to the wire 20 from the direction opposite the retaining edge 497, e.g. from the closed side, that could otherwise cause the wire's 20 dislodgement or migration towards the opening side of the torque device 400 or 500. Such unintentional lateral movement could be caused by an operator's careless handling, for example, by dropping or bumping or otherwise disturbing torque device 400 or 500 with inserted guidewire 20 during deployment. Said wire captivating means is defined as any combination of one or more wire captivating clamps 470 or 470B that is comprised at least of the wire clamping pad 460, which further includes the wire captivating notch 490, which further includes a wire captivating notch retaining edge 497, a wire captivating notch ceiling 494, the notch 490 further having a height 495 and length 496, and facing a clamping surface 161. Another embodiment may further include a wire positioning guide 499. More simply, the wire captivating means is for the purpose of interfering with the unintentional or accidental lateral movement of a guidewire 20 during its deployment to prevent its unintentional or accidental movement or dislodgement out of said torque device 400 or 500. In certain embodiments, one or more elements of the clamps 470 or 470B may be of limited-width. The wire captivating means shall be construed only as described and understood herein and restricted to the apparatus and functions described, in their entirety, in this Specification and the Figures.

Material that may be used in forming the frames 100, 100A, 100B, 401B, 501A and 501B include metals, rigid and semi-rigid thermoplastics and others well-known to those skilled in the art. Examples of thermoplastics include nylon, glass-filled nylon, ABS, acetal, copolyester, polypropylene, PVC, or polycarbonate, though any similar plastic may be used. Elastomers may alternatively be used. A hard, rigid material, for example a glass-filled nylon, acetal or a polycarbonate, may be preferred at least for the clamping surfaces 161 and 461 or wire captivating notch 490, since they will then resist deformation during retention of a guidewire 20. Such deformation is disadvantageous since this would, in turn cause the surfaces 161 and 461 or notch ceiling 494 to conform to the circumference of the guidewire 20 during deployment, thus increasing wire-contacting surface area and reducing applied force and local pressure. Materials used exclude incorporation, addition or inclusion of abrasives.

The frames 100, 100A, 100B 401B, 501A and 501B can be formed by means of injection molding, 3-D printing, milling or other methods well-known to those skilled in the art. Although in a preferred embodiment they are formed as unitary structures, they may alternatively be composed of two or more discrete components that are assembled together during their manufacture.

The spring 300 is expected to be made of a metal, for example, steel, more particularly medical grade steel, though other materials, including thermoplastics or thermoplastic elastomers, may be used instead to form a C spring or other structure to apply clamping force. Although other spring shapes may be used, a “C”-shaped spring requires a minimum of material, is easily formed, is easily assembled into the frames 100A, 100B, 401B, 501A or 501B and thus avoids the higher material, design, manufacturing and labor cost of, e.g. coil springs.

The present invention is intended for use with any guidewire 20 used for medical purposes, including, more particularly, coated guidewires. For purposes of clarity and interpreting the Claims presented herein, the following terms shall have the meanings clearly associated with their descriptions, in their entirety, in this Specification and in the Figures: pivoting axis P, pivotable movement M, longitudinal axis L, clamping body 112, pivoting assembly element, and limited-width.

Though the Figures show shapes and relative dimensions of the elements of the devices 10, 400 and 500 in certain embodiments, alternative embodiments may include similar elements that have different shapes or relative dimensions, e.g. a spinner handle 130 may be wider and longer and in an oblate (instead of circular) cross-section compared to what is shown in the Figures herein. Alternative embodiments of the present invention may include combinations of features described herein that are different than explicitly described or shown, and these are still within the scope of the present invention. Specifically, each of the embodiments, i.e. torque devices 10, 10′, 400 and 500, include some features and characteristics that differ from each other and it is understood that different combinations of such features and characteristics from one or more of these embodiments may result in devices different from those explicitly presented herein and these are nonetheless included in the present invention. For example, the wire captivating notch 490 or wire catch 520 may be included in the device 10, or the wire clamp 160 or interlocking key pivot 250A may be included in device 500.

It will be further understood that the present invention is not limited to the details described and illustrated herein and that other changes in form and detail may be made without departing from the spirit and scope of the invention as defined in the appended claims. It is further intended that any other embodiments of the present invention that result from any changes in application or method of use or operation, fabrication, configuration, dimension, shape, method of manufacture, shape, size, or material, which are not specified within the detailed written description or illustrations contained herein yet would be understood by one skilled in the art, are within the scope of the present invention.

We claim:

1. A torque device apparatus for manipulating a guidewire used by a clinician in a medical procedure that includes at least two frames assembled together only by one or more springs, each frame including at least a clamping body member and a wire clamping pad, wherein:
the clamping body members face each other in the assembled apparatus to form a clamping body;
the assembled apparatus further includes at least one pivoting assembly element;
the wire clamping pads further include wire clamping surfaces that face each other in the assembled apparatus, these clamping pads and clamping surfaces thereby forming a wire clamp; and,
the assembled apparatus opens in a pivotable movement so that a guidewire may be inserted from the side when opened and retained therein when closed.

2. The apparatus of claim 1 wherein the frames each further include a spinner handle, the interior surfaces of which face each other in the assembled apparatus thereby forming a first spinner that is used by a clinician for grasping and manipulating the apparatus.

3. The apparatus of claim 1 wherein the one or more springs are more particularly formed in a C shape.

4. The apparatus of claim 1 wherein the at least one pivoting assembly element is more particularly one of a centric locking mechanism, an interlocking pivot, or an interlocking key pivot wherein:
the centric locking mechanism comprises at least a pivot tab and a pivot cavity, the pivot tab matably fitting inside the pivot cavity, the frames joined together only by the one or more springs;
the interlocking pivot comprises at least an elongate pivot bar and elongate pivot socket, the pivot bar matably fitting inside the pivot socket, the frames joined together only by the one or more springs;
the interlocking key pivot comprises at least the interlocking pivot with a pivot tab matably fitting inside a pivot cavity that is embedded in the interlocking pivot, the frames joined together only by the one or more springs.

5. The apparatus of claim 1, wherein at least one of the wire clamping pads more particularly includes a wire captivating notch to form a wire captivating clamp, the wire captivating notch further including a wire captivating notch ceiling and a wire captivating notch retaining edge, the wire captivating notch having a wire captivating notch height, wherein said wire captivating notch secures a retained guidewire by limiting unintentional movement or dislodgement during deployment.

6. The apparatus of claim 1 wherein the clamping body further includes at least one selected from the group comprising a backstop, pivoting assembly element and a wire catch.

7. The apparatus of claim 1 wherein at least one wire clamp includes limited-width wire clamping surfaces.

8. The apparatus of claim 2 wherein the first spinner further includes at least one selected from the group comprising a backstop, a wire catch, a wire clamp, a wire captivating clamp, and a pivoting assembly element.

9. The apparatus of claim 5, wherein at least one of the wire clamping surfaces, wire captivating notch ceiling, and wire captivating notch retaining edge are of limited-width.

10. The apparatus of claim 8 that further includes a second spinner at the end of the clamping body opposite the first spinner.

11. The apparatus of claim 8 wherein the wire clamp is more particularly a wire captivating clamp.

12. The apparatus of claim 10 wherein the second spinner further includes at least one selected from the group comprising a backstop, wire catch, a wire clamp, a wire captivating clamp, and a pivoting assembly element.

13. A wire captivating clamp, which is part of a side-loading torque device apparatus for manipulating a guidewire used by a clinician in a medical procedure, that includes facing clamping pads at least one of which further includes a wire captivating notch, said notch further including a wire captivating notch ceiling, a wire captivating notch retaining edge, and a wire captivating notch height, the wire captivating notch securing a retained guidewire by limiting unintentional lateral movement or dislodgement during deployment.

14. The wire captivating clamp of claim 13, wherein at least one of the wire captivating ceiling, the wire captivating notch retaining edge and the clamping surface are of limited-width.

15. The wire captivating clamp of claim 13 that further includes a wire positioning guide on at least one of the clamping pad and wire captivating notch ceiling.

16. A side-loading torque device apparatus for manipulating a guidewire used by a clinician in a medical procedure that includes at least:
a clamping means for the purpose of securely grasping a guidewire so as to avoid its rotational and longitudinal movement inside of and relative to said apparatus;
a hinging securement means and a pivoting means for the purposes of enabling positioning and assembly of the frames of said apparatus, and for enabling and permitting only pivotable movement of one frame against the other around a pivoting axis; and,
a guidewire manipulation means for the purpose of enabling a clinician to grasp said apparatus, and to manipulate said apparatus so as to rotate, and advance and withdraw the guidewire retained therein during its deployment in a patient.

17. The apparatus of claim 16 that further includes at least a wire captivating means for the purpose of interfering with the unintentional lateral movement of a guidewire during deployment to prevent its accidental movement or dislodgement out of said apparatus.

18. A hinging, spring-driven, side-loading guidewire torque device apparatus for manipulating a guidewire used by a clinician in a medical procedure that is assembled from at least two frames joined together only by one or more springs, and which includes a wire clamp, at least one selected from the group comprising a centric locking mechanism, an interlocking pivot and an interlocking key pivot, and which excludes axles, pins or living hinges as elements for connecting said frames, and which further enables: loading of the guidewire from the side instead of threading it though an end; securely retaining such guidewire within the device; and, advancing and rotating such guidewire during a medical procedure by a clinician's manual manipulation of the spinner.

19. A hinging securement means for the purposes of enabling positioning and assembly of the frames of a side-loading guidewire torque device, and for enabling and permitting only pivotable movement of one frame against the other around a pivoting axis.

20. A wire captivating means for the purpose of for the purpose of interfering with the unintentional lateral movement of a guidewire during deployment to prevent its accidental movement or dislodgement out of said apparatus.
